SEQUENCE LISTING (227 sequences)

SEQ ID NO: 1 - protein, 122 aa, Homo sapiens
EVQLVQSGAEVKKPGSSVKVSCKASGGTFSSYAISWVRQAPGQGLEWMGGIIPIFGTADYAQKFQGRATITADESTSTAYMELSSLRSEDTAVYYCARDYDFWSGYYAGDVWGQGTTVTVSS

SEQ ID NO: 2 - protein, 117 aa, Homo sapiens
QVQLVESGGGVVQPGRSLRLSCAASGFTFSTFPMHWVRQAPGKGLEWVAVIDYEGINKYYADSVKGRFTISRDNSKNTLYLQMNSLRAEDTAVYYCAGGSYYVPDYWGQGTLVTVSS

SEQ ID NO: 3 - protein, 117 aa, Homo sapiens
QLQLQESGGGVVQPGRSLRLSCAASGFTFSSFPMMWIRQAPGKGLEWVASISYDGSNKYYADSVKGRFTISRDNSKNTLYLQMNSLRAEDTAVYYCAGGSYYVPDYWGQGTLVTVSS

SEQ ID NO: 4 - protein, 117 aa, Homo sapiens
QLQLQESGGGVVQPGRSLRLSCAASGFTFSRNQMWWVRQAPGKGLEWVAVISIDQSVKYYADSVKGRFTISRDNSKNTLYLQMNSLRAEDTAVYYCAGGSYYVPDYWGQGTLVTVSS

SEQ ID NO: 5 - protein, 117 aa, Homo sapiens
QLQLQESGGGVVQPGRSLRLSCAASGFTFSTFPMHWVRQAPGKGLEWVAVIDYEGINKYYADSVKGRFTISRDNSKNTLYLQMNSLRAEDTAVYYCAGGSYYVPDYWGQGTLVTVSS

SEQ ID NO: 6 - protein, 117 aa, Homo sapiens
QLQLQESGGGVVQPGRSLRLSCAASGFTFSESAMHWVRQAPGKGLEWVAAISYDGSNKYYADSVKGRFTISRDNSKNTLYLQMNSLRAEDTAVYYCAGGSYYVPDYWGQGTLVTVSS

SEQ ID NO: 7 - protein, 117 aa, Homo sapiens
QLQLQESGGGVVQPGRSLRLSCAASGFTFSVFAMQWVRQAPGKGLEWVAAISYDGDNKYYADSVKGRFTISRDNSKNTLYLQMNSLRAEDTAVYYCAGGSYYVPDYWGQGTLVTVSS

SEQ ID NO: 8 - protein, 117 aa, Homo sapiens
QLQLQESGGGVVQPGRSLRLSCAASGFTFSERQMWWVRQAPGKGLEWVAVISNDTSSKYYADSVKGRFTISRDNSKNTLYLQMNSLRAEDTAVYYCAGGSYYVPDYWGQGTLVTVSS

SEQ ID NO: 9 - protein, 117 aa, Homo sapiens
QLQLQESGGGVVQPGRSLRLSCAASGFTFSERQMWWVRQAPGKGLEWVAVISHDGSTKYYADSVKGRFTISRDNSKNTLYLQMNSLRAEDTAVYYCAGGSYYVPDYWGQGTLVTVSS

SEQ ID NO: 10 - protein, 117 aa, Homo sapiens
QLQLQESGGGVVQPGRSLRLSCAASGFTFSSRQMWWVRPAPGKGLEWVAVISHDASAKYYADSVKGRFTISRDNSKNTLYLQMNSLRAEDTAVYYCAGGSYYVPDYWGQGTLVTVSS

SEQ ID NO: 11 - protein, 117 aa, Homo sapiens
QLQLQESGGGVVQPGRSLRLSCAASGFTFSVISMQWVRQAPGKGLEWVASISYDGSNKYYADSVKGRFTISRDNSKNTLYLQMNSLRAEDTAVYYCAGGSYYVPDYWGQGTLVTVSS

SEQ ID NO: 12 - protein, 117 aa, Homo sapiens
QLQLQESGGGVVQPGRSLRLSCAASGFTFSTFPMHWVRQAPGKGLEWVAAISYAGSNKYYADSVKGRFTISRDNSKNTLYLQMNSLRAEDTAVYYCAGGSYYVPDYWGQGTLVTVSS

SEQ ID NO: 13 - protein, 117 aa, Homo sapiens
QLQLQESGGGVVQPGRSLRLSCAASGFTFSTLPMHWVRQAPGKGLEWVAVISYNGENKYYADSVKGRFTISRDNSKNTLYLQMNSLRAEDTAVYYCAGGSYYVPDYWGQGTLVTVSS

SEQ ID NO: 14 - protein, 117 aa, Homo sapiens
QLQLQESGGGVVQPGRSLRLSCAASGFTFSTLPMHWVRQAPGKGLEWVAVISYDGSNKYYADSVKGRFTISRDNSKNTLYLQMNSLRAEDTAVYYCAGGSYYVPDYWGQGTLVTVSS

SEQ ID NO: 15 - protein, 117 aa, Homo sapiens
QLQLQESGGGVVQPGRSLRLSCAASGFTFSERQMWWVRQAPGKGLEWVAVISNDSSQKYYADSVKGRFTISRDNSKNTLYLQMNSLRAEDTAVYYCAGGSYYVPDYWGQGTLVTVSS

SEQ ID NO: 16 - protein, 117 aa, Homo sapiens
QLQLQESGGGVVQPGRSLRLSCAASGFTFSTMSMQWVRQAPGKGLEWVASISYDGSNKYYADSVKGRFTISRDNSKNTLYLQMNSLRAEDTAVYYCAGGSYYVPDYWGQGTLVTVSS

SEQ ID NO: 17 - protein, 117 aa, Homo sapiens
QLQLQESGGGVVQPGRSLRLSCAASGFTFSTLSMGWVRQAPGKGLEWVAWISYDGSNKYYADSVKGRFTISRDNSKNTLYLQMNSLRAEDTAVYYCAGGSYYVPDYWGQGTLVTVSS

SEQ ID NO: 18 - protein, 117 aa, Homo sapiens
QLQLQESGGGVVQPGRSLRLSCAASGFTFSTSAMQWVRQAPGKGLEWVAVIGYDGANKYYADSVKGRFTISRDNSKNTLYLQMNSLRAEDTAVYYCAGGSYYVPDYWGQGTLVTVSS

SEQ ID NO: 19 - protein, 117 aa, Homo sapiens; identical to SEQ ID NO: 14

SEQ ID NO: 20 - protein, 117 aa, Homo sapiens
QLQLQESGGGVVQPGRSLRLSCAASGFTFSSYAMHWVRQAPGKGLEWVAAISYDGRNKYYADSVKGRFTISRDNSKNTLYLQMNSLRAEDTAVYYCAGGSYYVPDYWGQGTLVTVSS

SEQ ID NO: 21 - protein, 117 aa, Homo sapiens
QLQLQESGGGVVQPGRSLRLSCAASGFTFSAGQMWWVRQAPGKGLEWVAVISHDESNKYYADSVKGRFTISRDNSKNTLYLQMNSLRAEDTAVYYCAGGSYYVPDYWGQGTLVTVSS

SEQ ID NO: 22 - protein, 117 aa, Homo sapiens
QLQLQESGGGVVQPGRSLRLSCAASGFTFSTYPMHWVRQAPGKGLEWVAVISYTGINKYYADSVKGRFTISRDNSKNTLYLQMNSLRAEDTAVYYCAGGSYYVPDYWGQGTLVTVSS

SEQ ID NO: 23 - protein, 117 aa, Homo sapiens
QLQLQESGGGVVQPGRSLRLSCAASGFTFSSRQMWWVRQAPGKGLEWVAVISHDASAKYYADSVKGRFTISRDNSKNTLYLQMNSLRAEDTAVYYCAGGSYYVPDYWGQGTLVTVSS

SEQ ID NO: 24 - protein, 117 aa, Homo sapiens
QLQLQESGGGVVQPGRSLRLSCAASGFTFSESAMHWVRQAPGKGLEWVAVISYSGMNKYYADSVKGRFTISRDNSKNTLYLQMNSLRAEDTAVYYCAGGSYYVPDYWGQGTLVTVSS

SEQ ID NO: 25 - protein, 117 aa, Homo sapiens; identical to SEQ ID NO: 21

SEQ ID NO: 26 - protein, 117 aa, Homo sapiens
QLQLQESGGGVVQPGRSLRLSCAASGFTFSESAMGWVRQAPGKGLEWVAWIGYDGQNKYYADSVKGRFTISRDNSKNTLYLQMNSLRAEDTAVYYCAGGSYYVPDYWGQGTLVTVSS

SEQ ID NO: 27 - protein, 117 aa, Homo sapiens
QLQLQESGGGVVQPGRSLRLSCAASGFTFSSQTMQWVRQAPGKGLEWVASISYDGENKYYADSVKGRFTISRDNSKNTLYLQMNSLRAEDTAVYYCAGGSYYVPDYWGQGTLVTVSS

SEQ ID NO: 28 - protein, 117 aa, Homo sapiens; identical to SEQ ID NO: 13

SEQ ID NO: 29 - protein, 117 aa, Homo sapiens
QLQLQESGGGVVQPGRSLRLSCAASGFTFSVQSMLWVRQAPGKGLEWVASIGYDGVNKYYADSVKGRFTISRDNSKNTLYLQMNSLRAEDTAVYYCAGGSYYVPDYWGQGTLVTVSS

SEQ ID NO: 30 - protein, 117 aa, Homo sapiens; identical to SEQ ID NO: 4

SEQ ID NO: 31 - protein, 117 aa, Homo sapiens
QLQLQESGGGVVQPGRSLRLSCAASGFTFSSFPMQWVRQAPGKGLEWVASIAYDGSNKYYADSVKGRFTISRDNSKNTLYLQMNSLRAEDTAVYYCAGGSYYVPDYWGQGTLVTVSS

SEQ ID NO: 32 - protein, 117 aa, Homo sapiens
QLQLQESGGGVVQPGRSLRLSCAASGFTFSMFAMHWVRQAPGKGLEWVAAISIDGSGKYYADSVKGRFTISRDNSKNTLYLQMNSLRAEDTAVYYCAGGSYYVPDYWGQGTLVTVSS

SEQ ID NO: 33 - protein, 117 aa, Homo sapiens
QLQLQESGGGVVQPGRSLRLSCAASGFTFSESPMFWVRQAPGKGLEWVAVISYTGYNKYYADSVKGRFTISRDNSKNTLYLQMNSLRAEDTAVYYCAGGSYYVPDYWGQGTLVTVSS

SEQ ID NO: 34 - protein, 117 aa, Homo sapiens
QLQLQESGGGVVQPGRSLRLSCAASGFTFSRHRMFWVRQAPGKGLEWVAGIGYWGWNKYYADSVKGRFTISRDNSKNTLYLQMNSLRAEDTAVYYCAGGSYYVPDYWGQGTLVTVSS

SEQ ID NO: 35 - protein, 117 aa, Homo sapiens
QLQLQESGGGVVQPGRSLRLSCAASGFTFSWRQMWWVRQAPGKGLEWVAVISHDGSGKYYADSVKGRFTISRDNSKNTLYLQMNSLRAEDTAVYYCAGGSYYVPDYWGQGTLVTVSS

SEQ ID NO: 36 - protein, 117 aa, Homo sapiens
QLQLQESGGGVVQPGRSLRLSCAASGFTFSSSQMWWVRQAPGKGLEWVAVISHDTSSKYYADSVKGRFTISRDNSKNTLYLQMNSLRAEDTAVYYCAGGSYYVPDYWGQGTLVTVSS

SEQ ID NO: 37 - protein, 117 aa, Homo sapiens
QLQLQESGGGVVQPGRSLRLSCAASGFTFSRQQMWWVRQAPGKGLEWVAVISLDPSIKYYADSVKGRFTISRDNSKNTLYLQMNSLRAEDTAVYYCAGGSYYVPDYWGQGTLVTVSS

SEQ ID NO: 38 - protein, 117 aa, Homo sapiens; identical to SEQ ID NO: 32

SEQ ID NO: 39 - protein, 117 aa, Homo sapiens
QLQLQESGGGVVQPGRSLRLSCAASGFTFSSIPMFWVRQAPGKGLEWVASISYNGENKYYADSVKGRFTISRDNSKNTLYLQMNSLRAEDTAVYYCAGGSYYVPDYWGQGTLVTVSS

SEQ ID NO: 40 - protein, 117 aa, Homo sapiens
QLQLQESGGGVVQPGRSLRLSCAASGFTFSESSMQWVRQAPGKGLEWVASIGYDGQNKYYADSVKGRFTISRDNSKNTLYLQMNSLRAEDTAVYYCAGGSYYVPDYWGQGTLVTVSS

SEQ ID NO: 41 - protein, 117 aa, Homo sapiens
QLQLQESGGGVVQPGRSLRLSCAASGFTFSVQSMQWVRQAPGKGLEWVAAIGYDGENKYYADSVKGRFTISRDNSKNTLYLQMNSLRAEDTAVYYCAGGSYYVPDYWGQGTLVTVSS

SEQ ID NO: 42 - protein, 117 aa, Homo sapiens; identical to SEQ ID NO: 6

SEQ ID NO: 43 - protein, 117 aa, Homo sapiens; identical to SEQ ID NO: 9

SEQ ID NO: 44 - protein, 117 aa, Homo sapiens
QLQLQESGGGVVQPGRSLRLSCAASGFTFSSFAMHWVRQAPGKGLEWVAVISYDGSNKYYADSVKGRFTISRDNSKNTLYLQMNSLRAEDTAVYYCAGGSYYVPDYWGQGTLVTVSS

SEQ ID NO: 45 - protein, 117 aa, Homo sapiens; identical to SEQ ID NO: 9

SEQ ID NO: 46 - protein, 117 aa, Homo sapiens
QLQLQESGGGVVQPGRSLRLSCAASGFTFSSLPMHWVRQAPGKGLEWVAAISYDGSNKYYADSVKGRFTISRDNSKNTLYLQMNSLRAEDTAVYYCAGGSYYVPDYWGQGTLVTVSS

SEQ ID NO: 47 - protein, 9 aa, Homo sapiens
YLEYRQVPG

SEQ ID NO: 48 - protein, 9 aa, Homo sapiens
EGDCAPEEK

SEQ ID NO: 49 - protein, 9 aa, Homo sapiens
FLWGPRALV

SEQ ID NO: 50 - protein, 9 aa, Homo sapiens
FRAVITKKV

SEQ ID NO: 51 - protein, 9 aa, Homo sapiens
KVSARVRFF

SEQ ID NO: 52 - protein, 9 aa, Homo sapiens
FAHPRKLLM

SEQ ID NO: 53 - protein, 9 aa, Homo sapiens
SVFAHPRKL

SEQ ID NO: 54 - protein, 9 aa, Homo sapiens
LRKYRAKEL

SEQ ID NO: 55 - protein, 9 aa, Homo sapiens
FREALSNKV

SEQ ID NO: 56 - protein, 9 aa, Homo sapiens
VYGEPRKLL

SEQ ID NO: 57 - protein, 9 aa, Homo sapiens
SVYWKLRKL

SEQ ID NO: 58 - protein, 9 aa, Homo sapiens
VRFLLRKYQ

SEQ ID NO: 59 - protein, 9 aa, Homo sapiens
FYGEPRKLL

SEQ ID NO: 60 - protein, 9 aa, Homo sapiens
RAPKRQRCM

SEQ ID NO: 61 - protein, 9 aa, Homo sapiens
LRKYRVKGL

SEQ ID NO: 62 - protein, 9 aa, Homo sapiens
SVFAHPRKL

SEQ ID NO: 63 - protein, 9 aa, Homo sapiens
VRIGHLYIL

SEQ ID NO: 64 - protein, 9 aa, Homo sapiens
FAHPRKLLT

SEQ ID NO: 65 - protein, 9 aa, Homo sapiens
IMPKTGFLI

SEQ ID NO: 66 - protein, 9 aa, Homo sapiens
VSARVRFFF

SEQ ID NO: 67 - protein, 9 aa, Homo sapiens
NYKHCFPEI

SEQ ID NO: 68 - protein, 9 aa, Homo sapiens
EYLQLVFGI

SEQ ID NO: 69 - protein, 9 aa, Homo sapiens
VMPKTGLLI

SEQ ID NO: 70 - protein, 9 aa, Homo sapiens
IMPKAGLLI

SEQ ID NO: 71 - protein, 9 aa, Homo sapiens
NWQYFFPVI

SEQ ID NO: 72 - protein, 9 aa, Homo sapiens
VVGNWQYFF

SEQ ID NO: 73 - protein, 9 aa, Homo sapiens
SYPPLHEWV

SEQ ID NO: 74 - protein, 9 aa, Homo sapiens
SYVKVLHHM

SEQ ID NO: 75 - protein, 9 aa, Homo sapiens
IFPKTGLLI

SEQ ID NO: 76 - protein, 9 aa, Homo sapiens
NYKRCFPVL

SEQ ID NO: 77 - protein, 9 aa, Homo sapiens
IMPKTGFLI

SEQ ID NO: 78 - protein, 9 aa, Homo sapiens
NWQYFFPVI

SEQ ID NO: 79 - protein, 9 aa, Homo sapiens
VVGNWQYFF

SEQ ID NO: 80 - protein, 9 aa, Homo sapiens
SYVKVLHHM

SEQ ID NO: 81 - protein, 9 aa, Homo sapiens
RFLLRKYQI

SEQ ID NO: 82 - protein, 9 aa, Homo sapiens
VYYTLWSQF

SEQ ID NO: 83 - protein, 9 aa, Homo sapiens
NYKRYFPVI

SEQ ID NO: 84 - protein, 9 aa, Homo sapiens
VYVGKEHMF

SEQ ID NO: 85 - protein, 9 aa, Homo sapiens
CYPSLYEEV

SEQ ID NO: 86 - protein, 9 aa, Homo sapiens
SMPKAALLI

SEQ ID NO: 87 - protein, 9 aa, Homo sapiens
SSISVYYTL

SEQ ID NO: 88 - protein, 9 aa, Homo sapiens
SYEKVINYL

SEQ ID NO: 89 - protein, 9 aa, Homo sapiens
CYPLIPSTP

SEQ ID NO: 90 - protein, 9 aa, Homo sapiens
LYDGMEHLI

SEQ ID NO: 91 - protein, 9 aa, Homo sapiens
LWGPITQIF

SEQ ID NO: 92 - protein, 9 aa, Homo sapiens
VYAGREHFL

SEQ ID NO: 93 - protein, 9 aa, Homo sapiens
YAGREHFLF

SEQ ID NO: 94 - protein, 9 aa, Homo sapiens
EYLQLVFGI

SEQ ID NO: 95 - protein, 9 aa, Homo sapiens
SYVKVLHHL

SEQ ID NO: 96 - protein, 9 aa, Homo sapiens
KVLEYVIKV

SEQ ID NO: 97 - protein, 9 aa, Homo sapiens
FLIIVLVMI

SEQ ID NO: 98 - protein, 9 aa, Homo sapiens
FLWGPRALA

SEQ ID NO: 99 - protein, 9 aa, Homo sapiens
YVIKVSARV

SEQ ID NO: 100 - protein, 9 aa, Homo sapiens
LVLGTLEEV

SEQ ID NO: 101 - protein, 9 aa, Homo sapiens
CILESLFRA

SEQ ID NO: 102 - protein, 9 aa, Homo sapiens
IMPKTGFLI

SEQ ID NO: 103 - protein, 9 aa, Homo sapiens
KVADLVGFL

SEQ ID NO: 104 - protein, 9 aa, Homo sapiens
YVLVTCLGL

SEQ ID NO: 105 - protein, 9 aa, Homo sapiens
KASESLQLV

SEQ ID NO: 106 - protein, 9 aa, Homo sapiens
KMVELVHFL

SEQ ID NO: 107 - protein, 9 aa, Homo sapiens
KIWEELSML

SEQ ID NO: 108 - protein, 9 aa, Homo sapiens
FLWGPRALI

SEQ ID NO: 109 - protein, 9 aa, Homo sapiens
KASEYLQLV

SEQ ID NO: 110 - protein, 9 aa, Homo sapiens
YILVTCLGL

SEQ ID NO: 111 - protein, 9 aa, Homo sapiens
GLLIIVLAI

SEQ ID NO: 112 - protein, 9 aa, Homo sapiens
LQLVFGIEV

SEQ ID NO: 113 - protein, 9 aa, Homo sapiens
HLYILVTCL

SEQ ID NO: 114 - protein, 9 aa, Homo sapiens
QLVFGIEVV

SEQ ID NO: 115 - protein, 9 aa, Homo sapiens
LLIIVLAII

SEQ ID NO: 116 - protein, 9 aa, Homo sapiens
GLVGAQAPA

SEQ ID NO: 117 - protein, 9 aa, Homo sapiens
FLWGPRALV

SEQ ID NO: 118 - protein, 9 aa, Homo sapiens
KVAELVHFL

SEQ ID NO: 119 - protein, 9 aa, Homo sapiens
YIFATCLGL

SEQ ID NO: 120 - protein, 9 aa, Homo sapiens
KIWEELSVL

SEQ ID NO: 121 - protein, 9 aa, Homo sapiens
ALSRKVAEL

SEQ ID NO: 122 - protein, 9 aa, Homo sapiens
GLLIIVLAI

SEQ ID NO: 123 - protein, 9 aa, Homo sapiens
FQAALSRKV

SEQ ID NO: 124 - protein, 9 aa, Homo sapiens
HLYIFATCL

SEQ ID NO: 125 - protein, 9 aa, Homo sapiens
LLIIVLAII

SEQ ID NO: 126 - protein, 9 aa, Homo sapiens
GLVGAQAPA

SEQ ID NO: 127 - protein, 9 aa, Homo sapiens
KVLHHMVKI

SEQ ID NO: 128 - protein, 9 aa, Homo sapiens
GNWQYFFPV

SEQ ID NO: 129 - protein, 9 aa, Homo sapiens
KVLEHVVRV

SEQ ID NO: 130 - protein, 9 aa, Homo sapiens
ALLEEEEGV

SEQ ID NO: 131 - protein, 9 aa, Homo sapiens
FLWGPRALA

SEQ ID NO: 132 - protein, 9 aa, Homo sapiens
KVDELAHFL

SEQ ID NO: 133 - protein, 9 aa, Homo sapiens
ALSNKVDEL

SEQ ID NO: 134 - protein, 9 aa, Homo sapiens
AVSSSSPLV

SEQ ID NO: 135 - protein, 9 aa, Homo sapiens
YTLVTCLGL

SEQ ID NO: 136 - protein, 9 aa, Homo sapiens
LLIIVLGTI

SEQ ID NO: 137 - protein, 9 aa, Homo sapiens
LVPGTLEEV

SEQ ID NO: 138 - protein, 9 aa, Homo sapiens
YIFATCLGL

SEQ ID NO: 139 - protein, 9 aa, Homo sapiens
FLWGPRALI

SEQ ID NO: 140 - protein, 9 aa, Homo sapiens
KIWEELSVL

SEQ ID NO: 141 - protein, 9 aa, Homo sapiens
FLIIILAII

SEQ ID NO: 142 - protein, 9 aa, Homo sapiens
KVAKLVHFL

SEQ ID NO: 143 - protein, 9 aa, Homo sapiens
IMPKTGFLI

SEQ ID NO: 144 - protein, 9 aa, Homo sapiens
FQAALSRKV

SEQ ID NO: 145 - protein, 9 aa, Homo sapiens
KASDSLQLV

SEQ ID NO: 146 - protein, 9 aa, Homo sapiens
GLVGAQAPA

SEQ ID NO: 147 - protein, 9 aa, Homo sapiens
KVLHHMVKI

SEQ ID NO: 148 - protein, 9 aa, Homo sapiens
GNWQYFFPV

SEQ ID NO: 149 - protein, 9 aa, Homo sapiens
GLMDVQIPT

SEQ ID NO: 150 - protein, 9 aa, Homo sapiens
LIMGTLEEV

SEQ ID NO: 151 - protein, 9 aa, Homo sapiens
ALDEKVAEL

SEQ ID NO: 152 - protein, 9 aa, Homo sapiens
KVLEHVVRV

SEQ ID NO: 153 - protein, 9 aa, Homo sapiens
FLWGPRALA

SEQ ID NO: 154 - protein, 9 aa, Homo sapiens
LMDVQIPTA

SEQ ID NO: 155 - protein, 9 aa, Homo sapiens
YILVTCLGL

SEQ ID NO: 156 - protein, 9 aa, Homo sapiens
KVAELVRFL

SEQ ID NO: 157 - protein, 9 aa, Homo sapiens
AIWEALSVM

SEQ ID NO: 158 - protein, 9 aa, Homo sapiens
RQAPGSDPV

SEQ ID NO: 159 - protein, 9 aa, Homo sapiens
GLLIIVLGM

SEQ ID NO: 160 - protein, 9 aa, Homo sapiens
FMFQEALKL

SEQ ID NO: 161 - protein, 9 aa, Homo sapiens
KVAELVHFL

SEQ ID NO: 162 - protein, 9 aa, Homo sapiens
FLWGSKAHA

SEQ ID NO: 163 - protein, 9 aa, Homo sapiens
ALLIIVLGV

SEQ ID NO: 164 - protein, 9 aa, Homo sapiens
KVINYLVML

SEQ ID NO: 165 - protein, 9 aa, Homo sapiens
ALSVMGVYV

SEQ ID NO: 166 - protein, 9 aa, Homo sapiens
YILVTALGL

SEQ ID NO: 167 - protein, 9 aa, Homo sapiens
VLGEEQEGV

SEQ ID NO: 168 - protein, 9 aa, Homo sapiens
VMLNAREPI

SEQ ID NO: 169 - protein, 9 aa, Homo sapiens
VIWEALSVM

SEQ ID NO: 170 - protein, 9 aa, Homo sapiens
GLMGAQEPT

SEQ ID NO: 171 - protein, 9 aa, Homo sapiens
SMLGDGHSM

SEQ ID NO: 172 - protein, 9 aa, Homo sapiens
SMPKAALLI

SEQ ID NO: 173 - protein, 9 aa, Homo sapiens
SLLKFLAKV

SEQ ID NO: 174 - protein, 9 aa, Homo sapiens
GLYDGMEHL

SEQ ID NO: 175 - protein, 9 aa, Homo sapiens
ILILSIIFI

SEQ ID NO: 176 - protein, 9 aa, Homo sapiens
MLLVFGIDV

SEQ ID NO: 177 - protein, 9 aa, Homo sapiens
FLWGPRAHA

SEQ ID NO: 178 - protein, 9 aa, Homo sapiens
GMLSDVQSM

SEQ ID NO: 179 - protein, 9 aa, Homo sapiens
KMSLLKFLA

SEQ ID NO: 180 - protein, 9 aa, Homo sapiens
FVLVTSLGL

SEQ ID NO: 181 - protein, 9 aa, Homo sapiens
KVTDLVQFL

SEQ ID NO: 182 - protein, 9 aa, Homo sapiens
VIWEALNMM

SEQ ID NO: 183 - protein, 9 aa, Homo sapiens
NMMGLYDGM

SEQ ID NO: 184 - protein, 9 aa, Homo sapiens
QIACSSPSV

SEQ ID NO: 185 - protein, 9 aa, Homo sapiens
ILILILSII

SEQ ID NO: 186 - protein, 9 aa, Homo sapiens
GILILILSI

SEQ ID NO: 187 - protein, 9 aa, Homo sapiens
GLEGAQAPL

SEQ ID NO: 188 - protein, 9 aa, Homo sapiens
AMASASSSA

SEQ ID NO: 189 - protein, 9 aa, Homo sapiens
KIIDLVHLL

SEQ ID NO: 190 - protein, 9 aa, Homo sapiens
KVLEYIANA

SEQ ID NO: 191 - protein, 9 aa, Homo sapiens
VLWGPITQL

SEQ ID NO: 192 - protein, 9 aa, Homo sapiens
GLLIIVLGV

SEQ ID NO: 193 - protein, 9 aa, Homo sapiens
VMWEVLSIM

SEQ ID NO: 194 - protein, 9 aa, Homo sapiens
FLFGEPKRL

SEQ ID NO: 195 - protein, 9 aa, Homo sapiens
ILHDKIIDL

SEQ ID NO: 196 - protein, 9 aa, Homo sapiens
FLWGPRAHA

SEQ ID NO: 197 - protein, 9 aa, Homo sapiens
AMDAIFGSL

SEQ ID NO: 198 - protein, 9 aa, Homo sapiens
YVLVTSLNL

SEQ ID NO: 199 - protein, 9 aa, Homo sapiens
HLLLRKYRV

SEQ ID NO: 200 - protein, 9 aa, Homo sapiens
GTLEELPAA

SEQ ID NO: 201 - protein, 9 aa, Homo sapiens
GLGCSPASI

SEQ ID NO: 202 - protein, 9 aa, Homo sapiens
GLITKAEML

SEQ ID NO: 203 - protein, 9 aa, Homo sapiens
MQLLFGIDV

SEQ ID NO: 204 - protein, 9 aa, Homo sapiens
KMAELVHFL

SEQ ID NO: 205 - protein, 9 aa, Homo sapiens
FLWGPRALV

SEQ ID NO: 206 - protein, 9 aa, Homo sapiens
KIWEELSVL

SEQ ID NO: 207 - protein, 9 aa, Homo sapiens
KASEYLQLV

SEQ ID NO: 208 - protein, 9 aa, Homo sapiens
ALSRKMAEL

SEQ ID NO: 209 - protein, 9 aa, Homo sapiens
YILVTCLGL

SEQ ID NO: 210 - protein, 9 aa, Homo sapiens
GLLGDNQIV

SEQ ID NO: 211 - protein, 9 aa, Homo sapiens
GLLIIVLAI

SEQ ID NO: 212 - protein, 9 aa, Homo sapiens
LQLVFGIEV

SEQ ID NO: 213 - protein, 9 aa, Homo sapiens
KVLHHLLKI

SEQ ID NO: 214 - protein, 9 aa, Homo sapiens
HLYILVTCL

SEQ ID NO: 215 - protein, 9 aa, Homo sapiens
QLVFGIEVV

SEQ ID NO: 216 - protein, 9 aa, Homo sapiens
LLIIVLAII

SEQ ID NO: 217 - protein, 9 aa, Homo sapiens
RIGHLYILV

SEQ ID NO: 218 - protein, 9 aa, Homo sapiens
GLVGAQAPA

SEQ ID NO: 219 - protein, 9 aa, synthetic construct
ALWGPRALV

SEQ ID NO: 220 - protein, 9 aa, Synthetic construct
FAWGPRALV

SEQ ID NO: 221 - protein, 9 aa, Synthetic construct
FLAGPRALV

SEQ ID NO: 222 - protein, 9 aa, Synthetic construct
FLWAPRALV

SEQ ID NO: 223 - protein, 9 aa, Synthetic construct
FLWGARALV

SEQ ID NO: 224 - protein, 9 aa, Synthetic construct
FLWGPAALV

SEQ ID NO: 225 - protein, 9 aa, Synthetic construct
FLWGPRAAV

SEQ ID NO: 226 - protein, 9 aa, Synthetic construct
FLWGPRALA

SEQ ID NO: 227 - protein, 9 aa, Synthetic construct
KVLEYVIKV

(19) United States
(12) Patent Application Publication
(10) Pub. No.: US 2025/0230403 A1
(43) Pub. Date: Jul. 17, 2025

(54) METHODS AND MEANS FOR ATTRACTING IMMUNE EFFECTOR CELLS TO TUMOR CELLS

(71) Applicant: APO-T B.V., OSS (NL)

(72) Inventors: Johan Renes, Oss (NL); Marta Kijanka, Oss (NL)

(21) Appl. No.: 19/095,440

(22) Filed: Mar. 31, 2025

Related U.S. Application Data
(63) Continuation of application No. 16/972,555, filed on Dec. 4, 2020.
(60) Provisional application No. 62/680,406, filed on Jun. 4, 2018.

(51) Int. Cl. C12N 5/00; A61K 39/00; A61K 40/11; A61K 40/31; A61K 40/42; C07K 14/725; C07K 16/28; C12N 5/0783

(52) U.S. Cl. CPC C12N 5/0093; A61K 40/11; A61K 40/31; A61K 40/4268; C07K 14/7051; C07K 16/2833; C12N 5/0636; A61K 2039/505; C07K 2317/31; C07K 2317/32; C07K 2317/569

(57) ABSTRACT

A method for eradicating tumor cells expressing on their surface an MHC-peptide complex comprising a peptide derived from MAGE, the method comprising contacting the cell with at least one immune effector cell through specific interaction of a specific binding molecule for the MHC-peptide complex. Described are bispecific immunoglobulins of which one arm specifically binds to a MHC-MAGE-derived peptide complex associated with aberrant cells, and the other arm specifically recognizes a target associated with immune effector cells. A pharmaceutical composition comprising such bispecific antibody and suitable diluents and/or excipients and a T cell comprising a T-cell receptor or a chimeric antigen receptor recognizing a MHC-peptide complex comprising a peptide derived from MAGE-A is described, as well as a method of producing a T cell comprising introducing into the T-cell nucleic acids encoding an α chain and a ß chain or a chimeric antigen receptor.

### CROSS-REFERENCE TO RELATED APPLICATIONS

This application is a continuation of co-pending U.S. patent application Ser. No. 16/972,555, filed Dec. 4, 2020, which is a national phase entry under 35 U.S.C. § 371 of International Patent Application PCT/NL2019/050323, filed Jun. 3, 2019, designating the United States of America and published as International Patent Publication WO 2019/235915 A1 on Dec. 12, 2019, which claims the benefit under Article 8 of the Patent Cooperation Treaty to U.S. Provisional Patent Application Ser. No. 62/680,406, filed Jun. 4, 2018, the disclosure of each of which is hereby incorporated herein in its entirety by this reference.

### STATEMENT ACCORDING TO 37 C.F.R. § 1.834(c)

Pursuant to 37 C.F.R. § 1.831 through 1.835, a Sequence Listing XML file entitled “3403.02-P16074.2US.xml,” 195 kilobytes in size, generated Mar. 26, 2025, has been submitted via EFS-Web in lieu of a paper copy. This Sequence Listing is hereby incorporated by reference in its entirety into the specification.

### TECHNICAL FIELD

This disclosure relates to the field of biotherapeutics. It also relates to the field of tumor biology. More, in particular, this disclosure relates to the field of molecules capable of attracting immune effector cells to aberrant cells in cancers. The disclosure also relates to such molecules targeting aberrant cells and attracting immune effector cells, while leaving normal cells essentially unaffected. More in particular, the disclosure relates to specific binding molecules comprising binding domains specific for at least two different binding sites, one being on the surface of aberrant cells, and the other on the surface of immune effector cells. The disclosure also relates to the use of these specific binding molecules in selectively killing cancer cells.

### BACKGROUND

Cancer is caused by oncogenic transformation in aberrant cells, which drives uncontrolled cell proliferation, leading to misalignment of cell-cycle checkpoints, DNA damage and metabolic stress. These aberrations should direct tumor cells toward an apoptotic path, which has evolved in multi-cellular animals as a means of eliminating abnormal cells that pose a threat to the organism. Indeed, most transformed cells or tumorigenic cells are killed by apoptosis. However, occasionally a cell with additional mutations that enable avoidance of apoptotic death survives, thus enabling its malignant progression. Thus, cancer cells can grow not only due to imbalances in proliferation and/or cell cycle regulation, but also due to imbalances in their apoptosis machinery; imbalances like, for example, genomic mutations resulting in non-functional apoptosis-inducing proteins or over-expression of apoptosis-inhibiting proteins form the basis of tumor formation. Fortunately, even cells that manage to escape the apoptosis signals this way when activated by their aberrant phenotype, are still primed for eradication from the organism. Apoptosis in these aberrant cells can still be triggered upon silencing or overcoming the apoptosis-inhibiting signals induced by mutations. Traditional cancer therapies can activate apoptosis, but they do so indirectly and often encounter tumor resistance. Direct and selective targeting of key components of the apoptosis machinery in these aberrant cells is a promising strategy for development of new anti-tumor therapeutics. Selective activation of the apoptosis pathway would allow for halting tumor growth and would allow for induction of tumor regression.

A disadvantage of many if not all anti-tumor drugs currently on the market or in development is that these drugs do not discriminate between aberrant cells and healthy cells. This non-specificity bears a challenging risk for drug-induced adverse events. Examples of such unwanted side effects are well known to the field: radiotherapy and chemotherapeutics induce cell death only as a secondary effect of the damage they cause to vital cellular components. Not only aberrant cells are targeted, though in fact most proliferating cells including healthy cells respond to the apoptosis-stimulating therapy. Therefore, a disadvantage of current apoptosis-inducing compounds is their non-selective nature, which reduces their potential.

Since the sixties of the last century, it has been proposed to use the specific binding power of the immune system (T cells and antibodies) to selectively kill tumor cells while leaving alone the normal cells in a patient's body. The introduction of monoclonal antibodies (mAb) has been a great step in bringing us closer toward personalized and more tumor-specific medicine. However, one of the major challenges, being the design of a therapy that is at the same time efficacious and truly cancer-specific, still remains unresolved. The majority of mAbs currently approved by the U.S. Food and Drug Administration and undergoing evaluation in clinical trials target cell surface antigens, more rarely to soluble proteins (Hong, C. W. et al. Cancer Res., 2012, 72(15): p. 3715-9; Ferrone, S., Sci. Transl. Med., 2011, 3(99): p. 99). These antigens represent hematopoietic differentiation antigens (e.g., CD20), glycoproteins expressed by solid tumors (e.g., EpCAM, CEA or CAIX), glycolipids (i.e., gangliosides), carbohydrates (i.e., Lewis Y antigen), stromal and extracellular matrix antigens (e.g., FAP), proteins involved in angiogenesis (e.g., VEGFR or integrins), and receptors involved in growth and differentiation signaling (e.g., EGFR, HER2 or IGF1R). For essentially all of these antigens, expression is associated with normal tissue as well. Thus, so far, selective killing of aberrant cells has been an elusive goal.

Proteins of the Melanoma Antigen Gene family (MAGE) were the first identified members of Cancer Testis antigens (CT). Their expression pattern is restricted to germ cells of immuno-privileged testis and placenta, as well as a wide range of malignant cells. Expression of CT antigens in cancer cells was shown to result in their uncontrolled growth, resistance to cell death, potential to migrate, grow at distant sites and the ability to induce growth of new blood vessels (Morten F. Gjerstorff et al. Oncotarget, 2015, 6(18): p. 15772-15787; Scanlan M. J., G. A. et al. Immunol. Rev., 2002, 188: p. 22-32). Due to their intracellular expression, MAGE proteins remain inaccessible targets until they undergo proteasomal degradation into short peptides in the cytoplasm. These peptides generated by the proteasome are then transported into endoplasmic reticulum where they are loaded onto the MHC class I molecules. Intracellularly processed MAGE-A-derived peptides can be used as an immunotherapy target once present on the cell membrane in complex with MHC class I molecules. The MHC molecules present the MAGE-derived peptides to specialized cells of the immune system. The few cells that do not express MHC class I molecules are the cells from testis and placenta. Therefore, normal cells that express MAGE protein do not have the MHC class I molecules, and the normal cells that have MHC class I molecules do not have the MAGE protein. The MAGE-derived peptides in context of MHC class I are, therefore, truly tumor-specific targets.

One of the subsets of immune effector cells are NK cells. Due to expression of CD16 on their surface, they are capable of recognition and binding of Fc parts of immunoglobulins. Upon binding of Fc region of an IgG to Fc receptor NK cells release cytotoxic factors that cause the death of the cell bound by the IgG. These cytotoxic factors include perforin and granzymes, a class of proteases, causing the lysis of aberrant cells. Such mode of attracting immune effector cells is referred to as “antibody-dependent cell-mediated cytotoxicity.” It is, of course, also possible, and in fact preferable, to have the second arm of the bispecific antibody recognize the CD16 and disable the Fc part of the bispecific antibody.

Attracting of immune effector cells, such as T cells, to aberrant cells can be done by (retroviral) introduction of chimeric T-cell receptors (cTCRs) or chimeric antibody receptors (CARs), providing specificity to markers expressed on the cell surface of aberrant cells. Chimeric TCRs have been so far generated by fusing an antibody-derived V_{H} and V_{L} chain to a TCR Cβ and Cα chain, respectively. T cells expressing these cTCRs have been described to show specific functionality in vitro (Gross, G. et al. Proceedings of the National Academy of Sciences, 1989, 86(24): p. 10024-10028). One of the advantages of this format over the CAR format would be that the intracellular signaling in T cells expressing cTCRs occurs via the natural CD3 complex, in contrast to the signaling in CAR-expressing T cells. Multiple clinical studies using TCR and CAR engineered T cells have shown promising results (Brentjens, R. J., et al. Science translational medicine, 2013, 5(177); Robbins, P. F., et al. Clinical Cancer Research, 2015. 21(5): p. 1019-1027; Porter, D. L., et al. Science translational medicine, 2015, 7(303)).

CARs represent the same principle of attracting immune effector cells to aberrant cells as chimeric TCRs, however, the molecule format differs. Three generations of CARs have been developed so far. First-generation CARs consist of antibody-derived V_{H} and V_{L} chains in a so-called single-chain (scFv), or Fab format, which are fused to a CD4 transmembrane domain and a signaling domain derived from one of the proteins within the CD3 complex (e.g., ζ, γ). To improve CAR T-cell function and persistence, second generation CARs were developed that contain one co-stimulatory endodomain derived from, for instance, CD28, OX40 (CD134) or 4-1BB (CD137). Third generation CARs harbor two co-stimulatory domains (Sadelain et al. Cancer discovery, 2013, 3(4): p. 388-398). For long, the use of CAR T-cell therapy has been restricted to small clinical trials, mostly enrolling patients with advanced blood cancers. The two lately approved by FDA therapies include one for the treatment of children with acute lymphoblastic leukemia (Kymriah by Novartis Pharmaceuticals Corporation) and the other for adults with advanced lymphomas (Yescarta by Kite Pharma, Inc.) Both of these employ CD19 molecule, also present on healthy B-cells, as tumor marker. Targeting solid tumors remains, however, a big challenge in the field of immuno-oncology. The main underlying reasons are low T-cell infiltration and the immunosuppressive environment that tumor cells create to evade immune cells.

Another possibility to attract immune effector cells to the tumor site is the use of bispecific antibodies. Bispecific antibodies are being developed as cancer therapeutics in order to (i) inhibit two cell surface receptors, (ii) block two ligands, (iii) cross-link two receptors or (iv) recruit immune cells that do not carry a Fc receptor (such cells are not activated by antibodies). Over time, several ways of production of bispecific antibodies have been developed. First, bispecific antibodies were produced either by reduction and re-oxidation of cysteines in the hinge region of monoclonal antibodies. Another option was to produce bispecific antibodies by fusion of two hybridomas. Such fusion resulted in formation of a quadroma, from which a mixture of IgG molecules is produced. Such production system provides, however, limited amount of actual bispecific molecules. Chimeric hybridomas, common light chains and recombinant proteins addressed the limitation of proper antibody light and heavy chain association in order to generate a bispecific molecule. The heavy-light chain pairing in chimeric quadromas is species restricted. Advances in the field of recombinant DNA technology opened up new opportunities regarding composition and production systems of bispecific antibodies. The correct bispecific antibody structure in a recombinant protein can be ensured by employing various strategies, such as, e.g., knobs-in-holes approach (one heavy chain is engineered with a knob consisting of relatively large amino acids, whereas the other is engineered with a hole consisting of relatively small amino acids) or connecting antibody fragments as peptide chains to avoid random association of the chains (e.g., employed in the BiTE approach). Bispecific antibodies can be categorized based on their structure into IgG-like molecules, which contain an Fc region, or non-IgG like that lack the Fc region. IgG-like bispecific molecules are bigger in size and have longer half-life in serum, whereas non-IgG-like antibodies have a smaller size that allows for better tumor penetration but exhibit a much shorter serum half-life. Availability of numerous formats of bispecific antibodies allows for modulation of their immunogenicity, effector functions and half-life.

Growing interest in immune-oncology resulted in the development of immune cell engaging antibodies. Examples of such bispecific antibodies, of which one binding arm recognizes a target expressed on the surface of a tumor cell and the second arm, an antigen present on the effector immune cells, such as, for example, CD3 on T cells have been described (Kontermann R. E., MAbs. 2012, 4(2):182-97; Chames et al. MAbs. 2009, 1(6):539-47; Moore et al. Blood, 2011, 117(17):4542-51). The so-called trio mAb CD3xEpcam bispecific antibody (“catumaxomab”), has been developed clinically and has been registered in Europe for palliative treatment of abdominal tumors of epithelial origin. Catumaxomab binds EpCAM-positive cancer cells with one antigen-binding arm and the T-cell antigen CD3 with the other (Chelius D. et al. MAbs. 2010, 2(3):309-19). In addition to the direction of T cells toward the EpCAM-positive cancer cells via the CD3 binding, this approach also facilitates the binding of other immune cells, e.g., natural killer cells and macrophages by the Fc domain of this antibody rendering this strategy bi-specific but tri-functional. The widespread application of this format is, however, prevented by its rodent nature, which induces anti-product immune responses upon repetitive dosing.

Alternative formats for molecules redirecting immune effector cells to cancer sites have been evaluated such as Dual-Affinity Re-Targeting (DART™) molecules that are developed by Macrogenics, Bispecific T cell Engager (BiTE®) molecules that were developed by Micromet, now Amgen (Sheridan C., Nat. Biotechnol. 2012 (30):300-1), Dual Variable Domain-immunoglobulin (DVD-Ig™) molecules that are developed by Abbott, and TANDAB® RECRUIT molecules that are developed by Affimed. Up to date the cancer related antigens targeted by these formats are not truly tumor-specific as in case of MAGE antigen. The CD3xCD19 BiTE®, blinatumomab, has demonstrated remarkable clinical efficacy in refractory non-Hodgkin lymphoma and acute lymphatic leukemia patients (Bargou R. et al. Science, 2008, 321(5891):974-7). One of the targets recognized by blinatumomab is CD19, a cell surface antigen expressed on both neoplastic and healthy B-cells. The results of Blinatumomab spiked the development of various molecules directing T-cell activity toward tumor sites. Some of these molecules, recognizing tumor associated but not tumor-specific targets such as EpCAM, CD33, ErbB family members (HER2, HER3, EGFR), death receptors (such as CD95 or CD63), proteins involved in angiogenesis (such as Ang-2 or VEGF-A) or PSMA, are currently undergoing clinical evaluation (Krishanumurthy A. et al. Pharmacol. Ther. 2018 May; 185:122-134).

There thus remains a need for effective specific binding molecules capable of recognizing a target exclusively accessible on the surface of aberrant cells and recruiting immune effector cells to such cells without being immunogenic.

### BRIEF DESCRIPTION OF THE DRAWINGS

FIGS. 1A and 1B: Specificity of A09 immunoglobulin was assessed in a flow cytometric assay employing a panel of cells of different origin. H1299 cells are HLA-A2-negative, MAGE-A-positive and serve as a negative control, H1299_A2/mMA are stably expressing HLA-A2/mMA complexes and serve as a positive control. U87 cells (HLA-A2-positive, MAGE-positive) are of glioblastoma origin, 911 cells (HLA-A2-positive, MAGE-negative) are derived from embryonic retinoblasts. (FIG. 1A) The binding of A09 was detected in a flow cytometric assay. The A09 IgG bound specifically to HLA-A2+, MAGE+ cells (U87, H1299_A2/mMA), however not to HLA-A2+, MAGE-cells (911) or HLA-A2-, MAGE+ cells (H1299). (FIG. 1B) The HLA-A2 expression status of used cell lines was assessed by flow cytometric staining using anti-HLA-A2-BB515 antibody.

FIGS. 2A and 2B: Transduced T cells express MAGE/HLA-A2-specific CAR on their surface. T cells transduced with scFv 4A6 CAR pMx-puro vector and control T cells transfected with pMx-puro vector were subjected to flow cytometric staining using tetramers of HLA-A2-MA3 (FLWGPRALV)-PE (SEQ ID NO:49). The tetramers were produced by mixing biotinylated HLA-A2-MA3 complexes with PE streptavidin at a molar ratio 5:1. Samples were incubated at 4° C., in the dark for 30 minutes. Detection of CD8-positive T cells was performed using the APC Mouse Anti Human CD8 (FIGS. 2A and 2B), whereas to detect the CD4 T cells, FITC Mouse Anti Human CD4 Antibody was used (FIG. 2A, bottom panel).

FIGS. 3A-1-3A-3 and 3B-1-3B-3: Granzyme B release as effect of T-cell activation. scFv 4A6 CAR T cells (B) or pMx-puro-RTV 014 T cells (FIGS. 3A-1-3A-3) were co-incubated with T2 cells pulsed with MA3 (relevant, FLWGPRALV (SEQ ID NO:49)) or MA1 (irrelevant) peptides. Ionomycin was used as a positive control for T-cell activation. Cells were stained extracellularly with anti-human CD8 (FIGS. 3A-1-3A-3, FIGS. 3B-1-3B-3 left column) and CD4 (FIGS. 3A-1-3A-3, FIGS. 3B-1-3B-3 right column), followed by intracellular staining with anti-human granzyme B (y axis: granzyme:PE, x axis: CD8/CD4).

FIGS. 4A-4F: Purification and specificity of bispecific molecules. (FIG. 4A) Bispecific molecules 4A6xCD3, A09xCD3 and CD19xCD3 were expressed in mammalian cells and purified from cell culture medium using Talon beads. Purity of elution fractions was assed using a stain free SDS-PAGE gel. (FIG. 4B) Purity of the bispecific molecules was assessed after de-salting step using stain-free SDS-PAGE. (FIG. 4C) 4A6xCD3 specifically binds HLA-A2/MA3,12 (black squares) and not HLA-A2/mMA (black circles) in ELISA on biotinylated peptide/HLA complexes. (FIG. 4D) 4A6xCD3 binds PBMCs from healthy donors (indicated by shift of MFI signal in bottom histogram when compared to upper histogram that serves as a background reference). Negative control molecule 4A6_SC_FV did not bind PBMC (as indicated by lack of shift in middle histogram when compared to upper histogram that serves as a background reference). (FIG. 4E) Alanine scanning analysis of 4A6xCD3 fine specificity. (FIG. 4F) Table showing amino acid sequences of peptides used in the alanine scanning experiment, as well as their predicted affinity to HLA-A2 molecule. Random peptide is used as a control peptide with high affinity toward HLA-A2. It is a negative control as 4A6xCD3 does not carry fine specificity toward this peptide/HLA complex.

FIGS. 5A-5F: T-cell activation by the bispecific molecule of the disclosure in context of H1299 cells expressing target MAGE-A-derived peptide/HLA complex. (FIG. 5A) 72-hour incubation of 500 ng/ml 4A6xCD3 (BiTE A) with H1299 expressing HLA-A2/MA3, 12 cells (Target A) and 72-hour incubation of 500 ng/ml A09xCD3 (BiTE B) with H1299 expressing HLA-A2/mMA cells (Target B) in presence of PBMC leads to increase of percentage of CD69-positive T cells. (FIG. 5B) 72-hour incubation of 500 ng/ml 4A6xCD3 (BiTE A) with H1299 expressing HLA-A2/MA3, 12 cells (Target A) and 72-hour incubation of 500 ng/ml A09xCD3 (BiTE B) in with H1299 expressing HLA-A2/mMA cells (Target B) in presence of PBMC leads to increase of percentage of CD25-positive T cells. (FIG. 5C) Representative histograms showing the mean fluorescent intensity (MFI) of T cells incubated with target cells as indicated in FIGS. 5A and 5B either without bispecific molecule 4A6xCD3 (upper histogram) or in presence of bispecific molecule 4A6xCD3 (middle histogram) or A09xCD3 (bottom histogram). (FIG. 5D) Dose-dependent increase in CD69 expression of T cells with increasing amounts of bispecific molecule. (FIG. 5E) Different target-to effector-cell ratios did not affect the percentage of CD69-positive T cells when incubated with either 4A6xCD3 on H1299 HLA-A2/MA3, 12 or A09xCD3 on H1299 HLA-A2/mMA cells. (FIG. 5F) Physical attraction of PBMC to H1299 expressing HLA-A2/MA3, 12 cells in presence of 4A6xCD3 after 24-hour incubation.

FIGS. 6A-6E: T-cell activation by the bispecific molecule of the disclosure in context of 911 cells expressing target MAGE-A-derived peptide/HLA complex. (FIG. 6A) 72-hour incubation of 500 ng/ml 4A6xCD3 with 911 cells expressing HLA-A2/MA3, 12 complex leads to increase of percentage of CD69-positive T cells. (FIG. 6B) 72-hour incubation of 500 ng/ml 4A6xCD3 with 911 cells expressing HLA-A2/MA3, 12 complex leads to increase of percentage of CD25-positive T cells. (FIG. 6C) Representative histograms showing the mean fluorescent intensity (MFI) of T cells incubated with target cells as indicated in FIGS. 6A and 6B either without bispecific molecule 4A6xCD3 (upper histograms) or in presence of bispecific molecule 4A6xCD3 (bottom histograms). (FIG. 6D) Different target-to effector-cell ratios did not affect the percentage of CD69-positive T cells when incubated with 4A6xCD3 in presence of 911 cells expressing HLA-A2/MA3, 12 complexes. (FIG. 6E) Representative images showing the decreased number of 911 cells expressing HLA-A2/MA3, 12 complexes upon 72-hour incubation with 4A6xCD3 and PBMCs.

FIGS. 7A, 7B: T-cell activation upon incubation with A09xCD3 and glioblastoma cells. (FIG. 7A) Specific increase in percentage of CD69-positive T cells was observed when PBMCs were incubated for 72 hours with 4A6xCD3 or A09xCD3 molecules in presence of U87 cells. (FIG. 7B) Representative histograms showing the mean fluorescent intensity (MFI) of CD69-positive T cells upon incubation with U87 cells either without bispecific molecule (upper histograms) or in presence of bispecific molecule (bottom histograms).

FIG. 8: Purification of bi-specific nanobody. Expressed nanobody present in the periplasmic fraction (P) after purification was no longer detectable in the flow through (F) and could be efficiently eluted from the purification beads (E). Elution fractions were pooled and desalted (DE).

FIG. 9: Specific binding of phage display selected Fab fragments to HLA-A2/mMA complexes (data shown in upper table). As a positive control, AH5 Fab (produced from pCES vector) and AH5 monoclonal IgG were used. Clones showing binding to HLA-A2/MA3 complexes (data shown in bottom table) are considered to not carry the desired fine specificity.

### DETAILED DESCRIPTION

Disclosed are means and methods to attract immune effector cells specifically to tumor cells. Also disclosed is a pharmaceutically active molecule that facilitates specific and effective induction of aberrant cell's death. In particular, disclosed is specifically and selectively targeting aberrant cells and induce apoptosis of these aberrant cells, leaving healthy cells essentially unaffected. MHC-1 peptide complexes on tumors of almost any origin are valuable targets, whereas MHC-2 peptide complexes are valuable targets on tumors of hematopoietic origin. In this application we will typically refer to MHC-I. Of course, in most of the embodiments, MHC-II may be used as well, so that MAGE/MHC-II peptide complexes are also part of the disclosure.

An aberrant cell is defined as a cell that deviates from its healthy normal counterparts. Aberrant cells are for example tumor cells, cells invaded by a pathogen such as a virus, and autoimmune cells. Thus, in one embodiment, provided is an immunoglobulin according to any of the aforementioned embodiments, wherein the MHC-peptide complex is specific for aberrant cells.

Thus, one embodiment disclosed herein provides a method for eradicating aberrant cells, in particular, tumor cells expressing on their surface a MHC-peptide complex comprising a peptide derived from MAGE comprising contacting the cell with at least one immune effector cell through specific interaction of a specific binding molecule for the MHC-peptide complex. According to this disclosure, the immune effector cells are brought into close proximity of aberrant cells. It is an important aspect of the disclosure that the target on the tumor cell, the MAGE/MHC-I peptide complex, is tumor-specific. Therefore, the effector cells attracted to the target will typically only induce cell death in aberrant cells. There are several ways of bringing immune effector cells, in particular, NK cells and T cells, in close proximity of the aberrant cells. Any such method that uses the MAGE/MHC-I peptide complex is in principle suitable for this disclosure. Preferred ones involve bispecific antibodies.

Another preferred method is to provide effector cells, in particular, T cells, with a specific binding molecule recognizing the MAGE/MHC-I peptide complex. Thus, the disclosure provides a binding molecule comprising a binding domain specifically recognizing a certain MHC-peptide complex exposed on the surface of an aberrant cell and a binding domain capable of attracting effector immune cells to this aberrant cell. As used herein, the term “specifically binds to a MHC-peptide complex” means that the molecule has the capability of specifically recognizing and binding a certain MHC-peptide complex, in the situation that a certain MHC-peptide complex is present in the vicinity of the binding molecule. Likewise, the term “capable of recruiting immune effector cells” means that the molecule has the capability of specifically recognizing and binding antigens specific to immune effector cells when the immune effector cells are present in the vicinity of the specific binding molecule.

The term “specifically binds” means, in accordance herewith, that the molecule is capable of specifically interacting with and/or binding to at least two amino acids of each of the target molecule as defined herein. The term relates to the specificity of the molecule, i.e., to its ability to discriminate between the specific regions of the target molecule. The specific interaction of the antigen-interaction-site with its specific antigen may result in an initiation of a signal, e.g., due to the induction of a change of the conformation of the antigen, an oligomerization of the antigen, etc. Further, the binding may be exemplified by the specificity of a “key-lock-principle.” Thus, specific motifs in the amino acid sequence of the antigen-interaction-site and the antigen bind to each other as a result of their primary, secondary or tertiary structure as well as the result of secondary modifications of the structure. The specific interaction of the antigen-interaction-site with its specific antigen may result as well in a simple binding of the site to the antigen.

The term “binding molecule” as used in accordance with this disclosure means that the bispecific construct does not or essentially does not cross-react with (poly)peptides of similar structures. Cross-reactivity of constructs under investigation may be tested, for example, by assessing binding of the constructs under conventional conditions (see, e.g., Harlow and Lane, Antibodies: A Laboratory Manual, Cold Spring Harbor Laboratory Press, 1988 and Using Antibodies: A Laboratory Manual, Cold Spring Harbor Laboratory Press, 1999) to the antigens of interest as well as to a number of more or less (structurally and/or functionally) closely related antigens. Only those constructs that bind to the antigens of interest but do not or do not essentially bind to any of the other antigens are considered specific for the antigen of interest.

If, according to the disclosure, a bispecific antibody is used, it is clear to the skilled person that any format of a bispecific antibody as disclosed herein before (such as BiTEs, DARTs etc.) are suitable. Typically, these formats will comprise a single polypeptide format or complexes of different polypeptide chains. These chains/polypeptides will typically comprise Vh, Vhh and/or Vl.

Some formats of bispecific antibodies, such as IgGs, include an Fc region. This is another binding moiety for immune effector cells. In formats where there is already an arm recognizing a target on the immune effector cell, this moiety may be disabled through known means.

In a preferred embodiment, a bispecific antibody comprises one arm specifically binding to a MHC-peptide complex comprising a peptide derived from MAGE associated with aberrant cells, and the other arm specifically recognizing a target associated with immune effector cells. Therefore, the disclosure provides bispecific antibody according to the disclosure, wherein the bispecific antibody is a human IgG, preferentially human IgG1 wherein the Fc part does not activate the Fc receptor.

The advantage of targeting MAGE-A has been described in our earlier application US-2015-0056198 incorporated herein by reference. Briefly, MAGE-A expression is restricted to, apart from testis and placenta, aberrant cells. Placenta and testis do not express classical MHC, de facto MAGE-A/MHC-I peptide complexes are tumor-specific targets. Because there are many possible combinations of MHC molecules and MAGE-A peptides it is possible to device alternating and/or combination therapies, which tackles the problem of tumor escape from therapy.

The term “immune effector cell” or “effector cell” as used herein refers to a cell within the natural repertoire of cells in the mammalian immune system that can be activated to affect the viability of a target cell. Immune effector cells include the following cell types: natural killer (NK) cells, T cells (including cytotoxic T cells), B cells, monocytes or macrophages, dendritic cells and neutrophilic granulocytes. Hence, the effector cell is preferably an NK cell, a T cell, a B cell, a monocyte, a macrophage, a dendritic cell or a neutrophilic granulocyte. According to the disclosure, recruitment of effector cells to aberrant cells means that immune effector cells are brought in close proximity to the aberrant target cells, such that the effector cells can kill (directly or indirectly by initiation of the killing process) the aberrant cells that they are recruited to.

Target antigens present on immune effector cells may include CD3, CD16, CD25, CD28, CD64, CD89, NKG2D and NKp46. The most preferred antigen on an immune effector cell is the CD3ε chain.

T cells are an example of immune effector cells that can be attracted by the specific binding molecule to the aberrant cells. CD3 is a well described marker of T cells that is specifically recognized by antibodies described in the prior art. Furthermore, antibodies directed against human CD3 are generated by conventional methods known in the art. The VH and VL regions of the CD3-specific domain are derived from a CD3-specific antibody, such as, e.g., but not limited to, OKT-3 or TR-66. In accordance with this disclosure, the VH and VL regions are derived from antibodies/antibody derivatives and the like, which are capable of specifically recognizing human CD3 epsilon in the context of other TCR subunits.

Methods of treating cancer with antibodies are well known in the art and typically include parenteral injection of efficacious amounts of antibodies, which are typically determined by dose escalation studies.

Disclosed is a bispecific antibody of the disclosure for use in treating cancer.

Another method of bringing together immune effector cells and aberrant cells is to provide immune effector cells with a cell surface associated molecule, typically a receptor. In this case, according to the disclosure, typically T cells are provided with a T-cell receptor and/or a chimeric antigen receptor that specifically recognizes MAGE-A/MHC-I peptide complexes. Therefore, disclosed is a method according to the disclosure wherein the specific binding molecule is a T-cell receptor and/or chimeric antigen receptor. These T cells are made by introducing into the T-cell nucleic acids encoding an a chain and a β chain or a chimeric antigen receptor.

The dosage of the specific binding molecules are established through animal studies, (cell-based) in vitro studies, and clinical studies in so-called rising-dose experiments. Typically, the doses of present-day antibody are 3-15 mg/kg body weight, or 25-1000 mg per dose, present day BiTe 28 μg/day dose infused over 48 hours and 2×10⁶−2×10⁸ CAR-positive viable T cells per kg body weight of present day CAR-T cells.

For administration to subjects, the specific binding molecule hereof must be formulated. Typically, the specific binding molecules will be given intravenously. For formulation simply water (saline) for injection may suffice. For stability reasons more complex formulations may be necessary. The disclosure contemplates lyophilized compositions as well as liquid compositions, provided with the usual additives.

Antibodies having the Vh domains given in SEQ ID NO:1 and SEQ ID NO:2 have been shown to have sufficient affinity and specificity to be used according to the disclosure.

Many binding domains able to specifically bind to MHC-peptide complexes are apparent to people of skill in the art. Immediately apparent are binding domains derived from the immune system, such as TCR domains and immunoglobulin (Ig) domains. Preferably, the domains encompass 100 to 150 amino acid residues. Preferably, the binding domains used for the disclosure are or are similar to variable domains (V_{H} or V_{L}) of antibodies. A good source for such binding domains are phage display libraries. Whether the binding domain of choice is actually selected from a library physically or whether only the information (sequence) is used is of little relevance. It is part of the disclosure that the binding molecule according to the disclosure preferably encompasses two or more variable domains of antibodies (“multispecificity”), linked through peptide bonds with suitable linker sequences. Classical formats of antibodies such as Fab, whole IgG and single chain Fv against MHC-peptide complexes are also within the disclosure.

As stated before, the binding domains selected according to the disclosure are preferably based on or derived from an immunoglobulin domain. The immunoglobulins (Ig) are suitable for the specific and selective localization attraction of immune effector cells to targeted aberrant cells, leaving healthy cells essentially unaffected. Immunoglobulins comprise immunoglobulin binding domains, referred to as immunoglobulin variable domains, comprising immunoglobulin variable regions. Maturation of immunoglobulin variable regions results in variable domains adapted for specific binding to a target binding site.

According to the present disclosure, the term “variable region” used in the context with Ig-derived antigen-interaction comprises fragments and derivatives of (poly)peptides that at least comprise one CDR derived from an antibody, antibody fragment or derivative thereof. It is envisaged by the disclosure that at least one CDR is preferably a CDR3, more preferably the CDR3 of the heavy chain of an antibody (CDR-H3).

Because the anticipated predominant use of the binding molecule hereof is in therapeutic treatment regimens meant for the human body, the immunoglobulins variable regions preferably have an amino-acid sequence of human origin. Humanized immunoglobulin variable regions, with the precursor antibodies encompassing amino acid sequences originating from other species than human, are also part hereof. Also, part hereof are chimeric molecules, comprising (parts of) an immunoglobulin variable region hereof originating from a species other than human.

The affinity of the specific binding molecule hereof for the two different target binding sites separately, preferably is designed such that Kon and Koff are very much skewed toward binding to both different binding sites simultaneously. Thus, in one embodiment hereof, the antibody according to any of the previous embodiments is a hetero-dimeric bi-specific immunoglobulin G or heavy-chain only antibody comprising two different but complementary heavy chains. The two different but complementary heavy chains may then be dimerized through their respective Fc regions. Upon applying preferred pairing biochemistry, hetero-dimers are preferentially formed over homo-dimers. For example, two different but complementary heavy chains are subject to forced pairing upon applying the “knobs-into-holes” CH3 domain engineering technology as described (Ridgway et al. Protein Engineering, 1996 (ref 14)). In a preferred embodiment hereof, the two different immunoglobulin variable regions in the bi-specific immunoglobulins hereof specifically bind with one arm to an MHC-peptide complex preferentially associated with aberrant cells, and to antigen present on immune effector cells.

Although the disclosure contemplates many different combinations of MHC and antigenic peptides the most preferred is the combination of MHC-1 and an antigenic peptide from a tumor related antigen presented by MHC-1. Because of HLA restrictions, there are many combinations of MHC-1-peptide complexes as well as of MHC-2-peptide rules include size limits on peptides that can be presented in the context of MHC, restriction sites that need to be present for processing of the antigen in the cell, anchor sites that need to be present on the peptide to be presented, etc. The exact rules differ for the different HLA classes and for the different MHC classes. We have found that MAGE-derived peptides are very suitable for presentation in an MHC context. An MHC-1 presentable antigenic peptide with the sequence Y-L-E-Y-R-Q-V-P-G (SEQ ID NO:47) in MAGE-A was identified, that is present in almost every MAGE-A variant (referred to as multi-MAGE peptide) and that will be presented by one of the most prevalent MHC-1 alleles in the Caucasian population (namely HLA-A0201). A second MAGE peptide that is presented by another MHC-1 allele (namely HLA-CW7) and that is present in many MAGE variants, like, for example, MAGE-A2,-A3,-A6 and-A12, is E-G-D-C-A-P-E-E-K (SEQ ID NO:48). These two combinations of MHC-1 and MAGE peptides together could cover 80% of the Caucasian population. Another MAGE peptide that is presented by the same MHC-I allele as the multi-MAGE peptide has a sequence F-L-W-G-P-R-A-L-V (SEQ ID NO:49) and is present in MAGE-A3 and MAGE-A12 proteins.

Thus, in one embodiment, provided is a list of MAGE-A-derived peptides presented in context of HLA-A0201, HLA-A2402 and HLA-C0701.

The disclosed embodiment is exemplified by the Examples below.

### EXAMPLE 1

Target binding sites suitable for specific and selective targeting of aberrant cells by specific binding molecules of the disclosure are MAGE-derived antigen peptides complexed with MHC molecules. Examples of T-cell epitopes of the MAGE-A protein, complexed with indicated HLA molecules, are provided below. Any combination of an HLA molecule complexed with a MAGE-derived T-cell epitope provides a specific target on aberrant cells for specific binding molecules hereof. Examples of suitable target MAGE-derived epitopes are peptides: FRAVITKKV (SEQ ID NO:50), KVSARVRFF (SEQ ID NO:51), FAHPRKLLM (SEQ ID NO:52), SVFAHPRKL (SEQ ID NO:53), LRKYRAKEL (SEQ ID NO:54), FREALSNKV (SEQ ID NO:55), VYGEPRKLL (SEQ ID NO:56), SVYWKLRKL (SEQ ID NO:57), VRFLLRKYQ (SEQ ID NO:58), FYGEPRKLL (SEQ ID NO:59), RAPKRQRCM (SEQ ID NO:60), LRKYRVKGL (SEQ ID NO:61), SVFAHPRKL (SEQ ID NO:62), VRIGHLYIL (SEQ ID NO:63), FAHPRKLLT (SEQ ID NO:64) presented via C0701; IMPKTGFLI (SEQ ID NO:65), VSARVRFFF (SEQ ID NO:66), NYKHCFPEI (SEQ ID NO:67), EYLQLVFGI (SEQ ID NO:68), VMPKTGLLI (SEQ ID NO:69), IMPKAGLLI (SEQ ID NO:70), NWQYFFPVI (SEQ ID NO:71), VVGNWQYFF (SEQ ID NO:72), SYPPLHEWV (SEQ ID NO:73), SYVKVLHHM (SEQ ID NO:74), IFPKTGLLI (SEQ ID NO:75), NYKRCFPVI (SEQ ID NO:76), IMPKTGFLI (SEQ ID NO:77), NWQYFFPVI (SEQ ID NO:78), VVGNWQYFF (SEQ ID NO:79), SYVKVLHHM (SEQ ID NO:80), RFLLRKYQI (SEQ ID NO:81), VYYTLWSQF (SEQ ID NO:82), NYKRYFPVI (SEQ ID NO:83), VYVGKEHMF (SEQ ID NO:84), CYPSLYEEV (SEQ ID NO:85), SMPKAALLI (SEQ ID NO:86), SSISVYYTL (SEQ ID NO:87), SYEKVINYL (SEQ ID NO:88), CYPLIPSTP (SEQ ID NO:89), LYDGMEHLI (SEQ ID NO:90), LWGPITQIF (SEQ ID NO:91), VYAGREHFL (SEQ ID NO:92), YAGREHFLF (SEQ ID NO:93), EYLQLVFGI (SEQ ID NO:94), SYVKVLHHL (SEQ ID NO:95) presented via A2402; KVLEYVIKV (SEQ ID NO:96), FLIIVLVMI (SEQ ID NO:97), FLWGPRALA (SEQ ID NO:98), YVIKVSARV (SEQ ID NO:99), LVLGTLEEV (SEQ ID NO:100), CILESLFRA (SEQ ID NO:101), IMPKTGFLI (SEQ ID NO:102), KVADLVGFL (SEQ ID NO:103), YVLVTCLGL (SEQ ID NO:104), KASESLQLV (SEQ ID NO:105), KMVELVHFL (SEQ ID NO:106), KIWEELSML (SEQ ID NO:107), FLWGPRALI (SEQ ID NO:108), KASEYLQLV (SEQ ID NO:109), YILVTCLGL (SEQ ID NO:110), GLLIIVLAI (SEQ ID NO:111), LQLVFGIEV (SEQ ID NO:112), HLYILVTCL (SEQ ID NO:113), QLVFGIEVV (SEQ ID NO:114), LLIIVLAII (SEQ ID NO:115), GLVGAQAPA (SEQ ID NO:116), FLWGPRALV (SEQ ID NO:117), KVAELVHFL (SEQ ID NO:118), YIFATCLGL (SEQ ID NO:119), KIWEELSVL (SEQ ID NO:120), ALSRKVAEL (SEQ ID NO:121), GLLIIVLAI (SEQ ID NO:122), FQAALSRKV (SEQ ID NO:123), HLYIFATCL (SEQ ID NO:124), LLIIVLAII (SEQ ID NO:125), GLVGAQAPA (SEQ ID NO:126), KVLHHMVKI (SEQ ID NO:127), GNWQYFFPV (SEQ ID NO:128), KVLEHVVRV (SEQ ID NO:129), ALLEEEEGV (SEQ ID NO:130), FLWGPRALA (SEQ ID NO:131), KVDELAHFL (SEQ ID NO:132), ALSNKVDEL (SEQ ID NO:133), AVSSSSPLV (SEQ ID NO:134), YTLVTCLGL (SEQ ID NO:135), LLIIVLGTI (SEQ ID NO:136), LVPGTLEEV (SEQ ID NO:137), YIFATCLGL (SEQ ID NO:138), FLWGPRALI (SEQ ID NO:139), KIWEELSVL (SEQ ID NO:140), FLIIILAII (SEQ ID NO:141), KVAKLVHFL (SEQ ID NO:142), IMPKTGFLI (SEQ ID NO:143), FQAALSRKV (SEQ ID NO:144), KASDSLQLV (SEQ ID NO:145), GLVGAQAPA (SEQ ID NO:146), KVLHHMVKI (SEQ ID NO:147), GNWQYFFPV (SEQ ID NO:148), GLMDVQIPT (SEQ ID NO:149), LIMGTLEEV (SEQ ID NO:150), ALDEKVAEL (SEQ ID NO:151), KVLEHVVRV (SEQ ID NO:152), FLWGPRALA (SEQ ID NO:153), LMDVQIPTA (SEQ ID NO:154), YILVTCLGL (SEQ ID NO:155), KVAELVRFL (SEQ ID NO:156), AIWEALSVM (SEQ ID NO:157), RQAPGSDPV (SEQ ID NO:158), GLLIIVLGM (SEQ ID NO:159), FMFQEALKL (SEQ ID NO:160), KVAELVHFL (SEQ ID NO:161), FLWGSKAHA (SEQ ID NO:162), ALLIIVLGV (SEQ ID NO:163), KVINYLVML (SEQ ID NO:164), ALSVMGVYV (SEQ ID NO:165), YILVTALGL (SEQ ID NO:166), VLGEEQEGV (SEQ ID NO:167), VMLNAREPI (SEQ ID NO:168), VIWEALSVM (SEQ ID NO:169), GLMGAQEPT (SEQ ID NO:170), SMLGDGHSM (SEQ ID NO:171), SMPKAALLI (SEQ ID NO:172), SLLKFLAKV (SEQ ID NO:173), GLYDGMEHL (SEQ ID NO:174), ILILSIIFI (SEQ ID NO:175), MLLVFGIDV (SEQ ID NO:176), FLWGPRAHA (SEQ ID NO:177), GMLSDVQSM (SEQ ID NO:178), KMSLLKFLA (SEQ ID NO:179), FVLVTSLGL (SEQ ID NO:180), KVTDLVQFL (SEQ ID NO:181), VIWEALNMM (SEQ ID NO:182), NMMGLYDGM (SEQ ID NO:183), QIACSSPSV (SEQ ID NO:184), ILILILSII (SEQ ID NO:185), GILILILSI (SEQ ID NO:186), GLEGAQAPL (SEQ ID NO:187), AMASASSSA (SEQ ID NO:188), KIIDLVHLL (SEQ ID NO:189), KVLEYIANA (SEQ ID NO:190), VLWGPITQI (SEQ ID NO:191), GLLIIVLGV (SEQ ID NO:192), VMWEVLSIM (SEQ ID NO:193), FLFGEPKRL (SEQ ID NO:194), ILHDKIIDL (SEQ ID NO:195), FLWGPRAHA (SEQ ID NO:196), AMDAIFGSL (SEQ ID NO:197), YVLVTSLNL (SEQ ID NO:198), HLLLRKYRV (SEQ ID NO:199), GTLEELPAA (SEQ ID NO:200), GLGCSPASI (SEQ ID NO:201), GLITKAEML (SEQ ID NO:202), MQLLEGIDV (SEQ ID NO:203), KMAELVHFL (SEQ ID NO:204), FLWGPRALV (SEQ ID NO:205), KIWEELSVL (SEQ ID NO:206), KASEYLQLV (SEQ ID NO:207), ALSRKMAEL (SEQ ID NO:208), YILVTCLGL (SEQ ID NO:209), GLLGDNQIV (SEQ ID NO:210), GLLIIVLAI (SEQ ID NO:211), LQLVFGIEV (SEQ ID NO:212), KVLHHLLKI (SEQ ID NO:213), HLYILVTCL (SEQ ID NO:214), QLVFGIEVV (SEQ ID NO:215), LLIIVLAII (SEQ ID NO:216), RIGHLYILV (SEQ ID NO:217), GLVGAQAPA (SEQ ID NO:218) presented via A0201.

A good source for selecting binding sites suitable for specific and selective targeting of aberrant cells hereof, is the NetMHC (on the WorldWideWeb at cbs.dtu.dk/services/NetMHC). The portal constitutes a prediction tool of peptide-MHC class I binding, upon uploading amino acid sequence of antigen of interest in context of MHC molecules comprising the indicated class of HLA.

### EXAMPLE 2

### A09 IgG Specifically Binds Human Aberrant Cells Presenting mMA Peptide Via HLA-A2

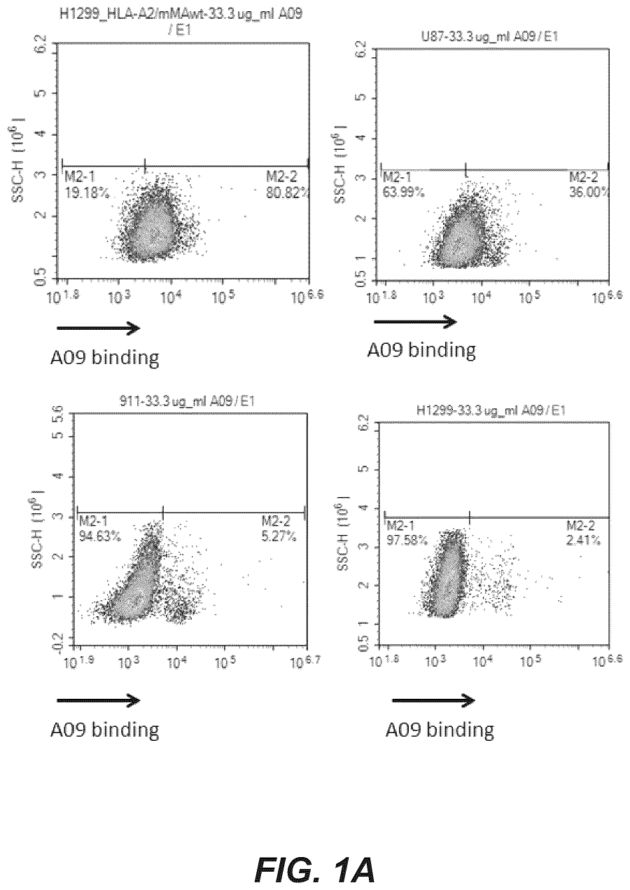

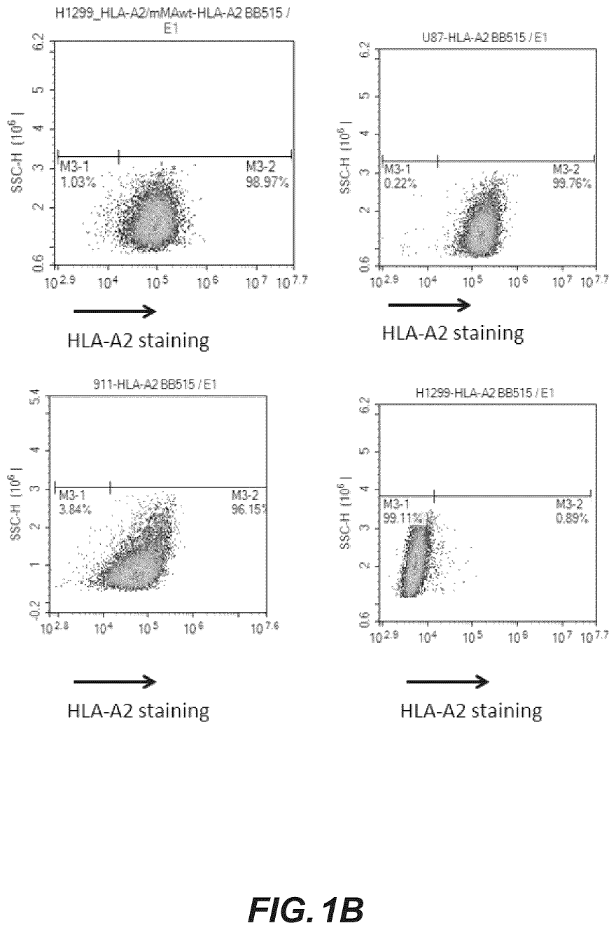

In order to confirm specificity of A09 IgG, the molecule was incubated with a panel of cell lines differing in their HLA-A2 and MAGE expression. Employed cell lines include non-small cell lung carcinoma H1299 (HLA-A2−, MAGE+), non-small cell lung carcinoma H1299_A2/mMA cells stably transfected with an expression construct of HLA-A2/mMA (HLA-A2+, MAGE+), glioblastoma cells U87 (HLA-A2+, MAGE+) and embryonic retinoblasts 911 (HLA-A2+, MAGE−). Briefly, the cells were spun down for 4 minutes at 450×g at 4° C. The supernatant was gently removed and the cell pellet resuspended in 100 μl of PBS+0.1% BSA per sample. Cells were transferred to the designated wells of a 96-well plate (100 μl/well) and spun down for 4 minutes at 450×g at 4° C. The supernatant was gently removed. The tested antibody in PBS+0.1% BSA was added to the cell pellet (20 μl/sample). The plate was shortly vortexed, in a gentle manner, to resuspend the cell pellet. Cells were incubated for 30 minutes at 2-8° C., upon which 200 μl of ice-cold PBS+0.1% BSA were added per well. Cells were washed by spinning down for 4 minutes at 450×g at 4° C. The supernatant was gently removed. Washing step was repeated. The primary detection antibody was diluted in PBS+0.1% BSA and added to the cell pellet (20 μl/sample). Samples were incubated for 30 minutes at 2-8° C. with goat anti human H+L IgG Alexa647 or mouse anti human HLA A2 BB515. At the end of the incubation, cells were washed twice as described before. Cells were fixed by resuspending the cell pellet in 200 μl of 1% PFA per sample at RT. The fluorescent signal was measured using Flow Cytometer. As shown in flow cytometric dot plots of FIG. 1A, the A09 antibody specifically recognized the multi MAGE peptide in complex with HLA-A0201. The expression of HLA-A0201 by H1299_A2/mMA cells, U87 cells and 911 cells was confirmed as shown in FIG. 1B.

### EXAMPLE 3

### Generation of T Cells Specifically Recognizing MAGE-A Peptide Presented in Context of HLA-A0201

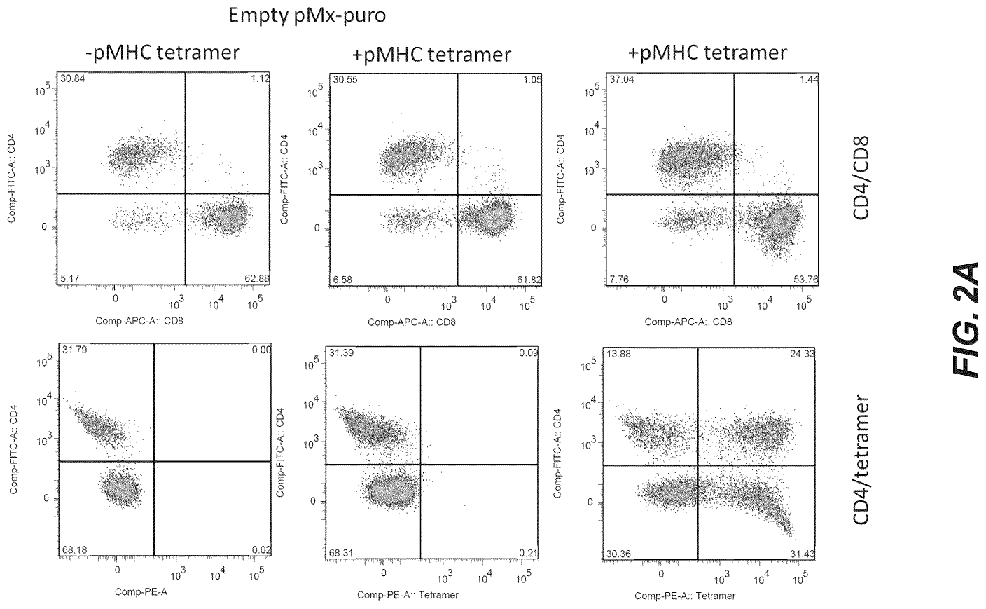

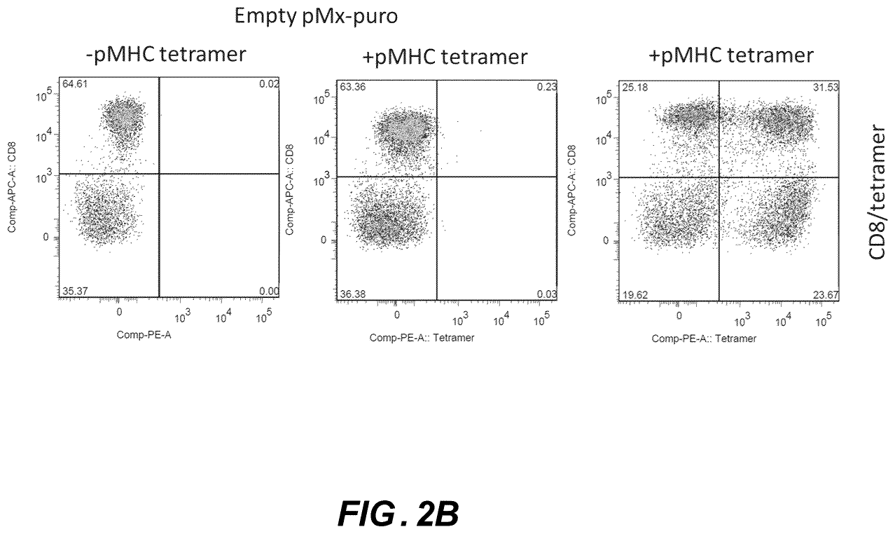

pMx-puro RTV014 vector and vector encoding scFv 4A6 CAR sequence were digested with BamHI and NotI. Digestion products were extracted from 1% agarose gel and purified using a DNA purification kit. The scFv 4A6 CAR purified fragments were ligated at 4° C. O/N with the purified pMx-puro RTV014 using the T4 ligase. Heat shock transformation of competent XL-I blue bacteria followed. Selection of transformed clones was based on ampicillin resistance (100 μg/ml). Plating of bacteria was performed on LB agar plates. Colonies were screened using restriction analysis. DNA was isolated using the Mini-prep DNA Isolation kit. Positive clones were grown in 100 ml LB+100 μg/ml ampicillin cultures. Phoenix Ampho cells were seeded at 1.2*10\hat{ }6 cells per 10 cm dish in DMEM (supplemented with 10% (V/V) fetal calf serum, 200 mM glutamine, 100 U penicillin, 100 μg/ml streptomycin), one day before transfection. Medium was refreshed 4 hours prior transfection. 800 μl serum free DMEM were mixed with 35μl of Fugene 6 reagent and incubated at RT for 5 minutes. 10 μg DNA (scFv 4A6 CAR pMx-puro RTV014) and 5 μg of each of the helper plasmids pHit60 and pColt-Galv were added to the mix. After incubating at RT for 15 minutes, the mix was added to the Phoenix Ampho cells. On the same day, PBMCs were thawed and seeded at a density of 2*10\hat{ }6 cells/well in a 24-well plate in 2 ml huRPMI containing 30 ng/ml of OKT-3 antibody and 600 U/ml IL-2. OKT-3 antibody was added to favor the proliferation of T cells in the PBMCs mixture. 24 hours later, the medium of the transfected Phoenix Ampho cells was replaced with huRPMI. The day after, the transduction was initiated. The viral supernatant was collected by centrifugation at 2000 rpm at 32° C. for 10 minutes. T cells were also collected by centrifugation at 1500 rpm at RT for 5 minutes. 2*10\hat{ }6 T cells were resuspended in 0.5 ml of viral supernatant with 5 μg/ml polybrene in a 24-well plate. Plates were spun at 2000 rpm for 90 minutes. T cells were cultured at 37° C. O/N. The next day, T cells were stimulated non-specifically with human CD3/CD28 beads. For specific stimulation of T cells, peptide-pulsed K562-HLA-A2-CD80 and 600 U/ml IL-2 were used. K562-HLA-A2-CD80 were pulsed with 10 μg peptide at 37° C. for 2 hours. Cells were then irradiated at 10,000 rad. 0.3*10\hat{ }6 of pulsed and irradiated K562-HLA-A2-CD80 cells were added to 0.5*10\hat{ }6 T cells in a final volume of 2 ml huRPMI/well in a 24-well plate. Detection of scFv 4A6 CAR was performed by flow cytometric staining using tetramers of HLA-A2-MA3 (FLWGPRALV)-PE (SEQ ID NO:49) (0.5 μl/sample). The tetramers were produced by mixing biotinylated HLA-A2-MA3 (FLWGPRALV (SEQ ID NO:49)) complexes with PE streptavidin at a molar ratio 5:1. Samples were incubated at 4° C., in the dark for 30 minutes. Flow cytometric staining shown in FIGS. 2A and 2B confirmed presence of 4A6 CAR-T cells.

### EXAMPLE 4

### Apoptosis Induction of Target-Expressing Cells upon Facilitating T Cells with Specific Binding Molecule of the Disclosure

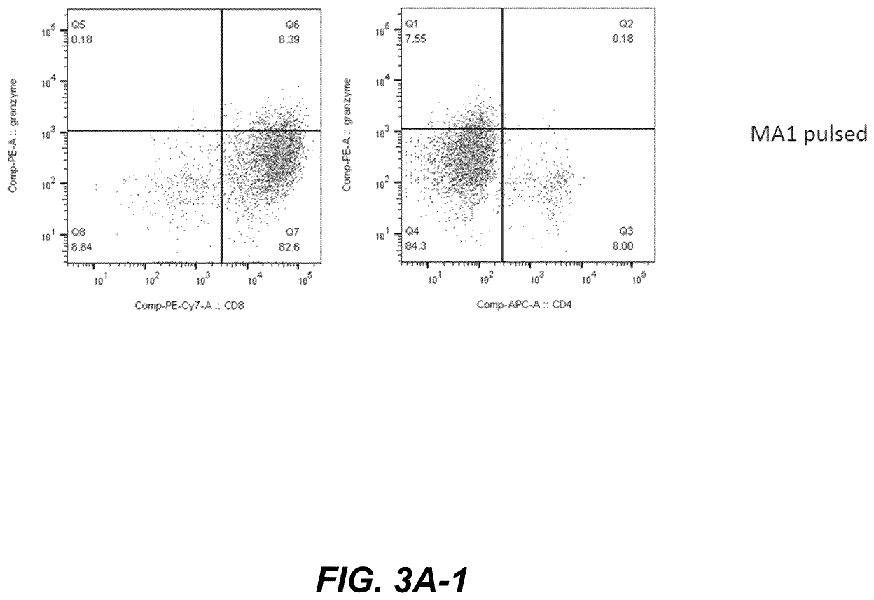

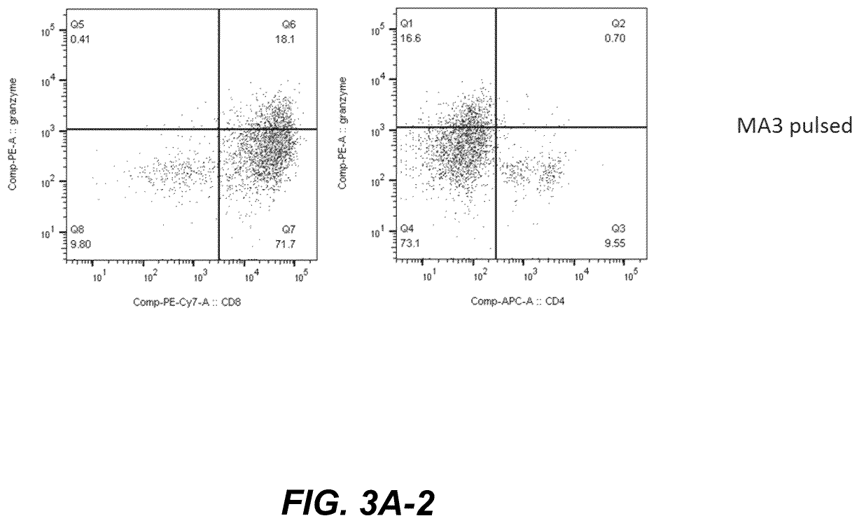

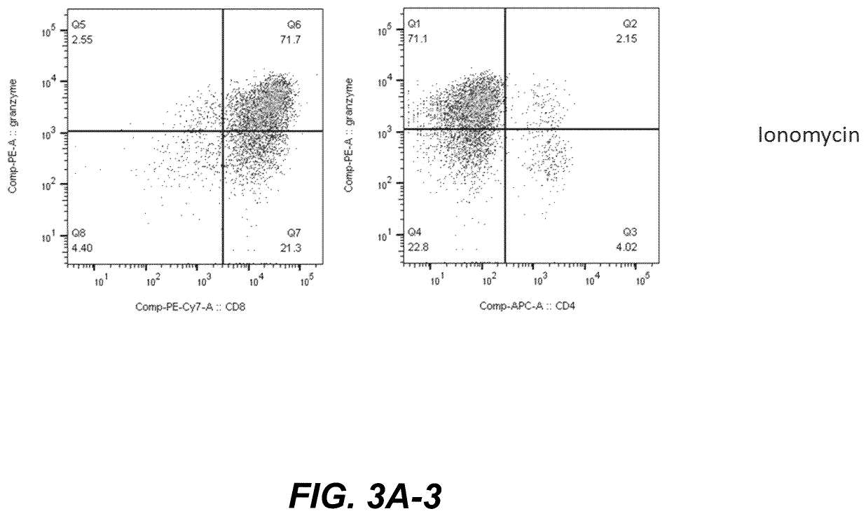

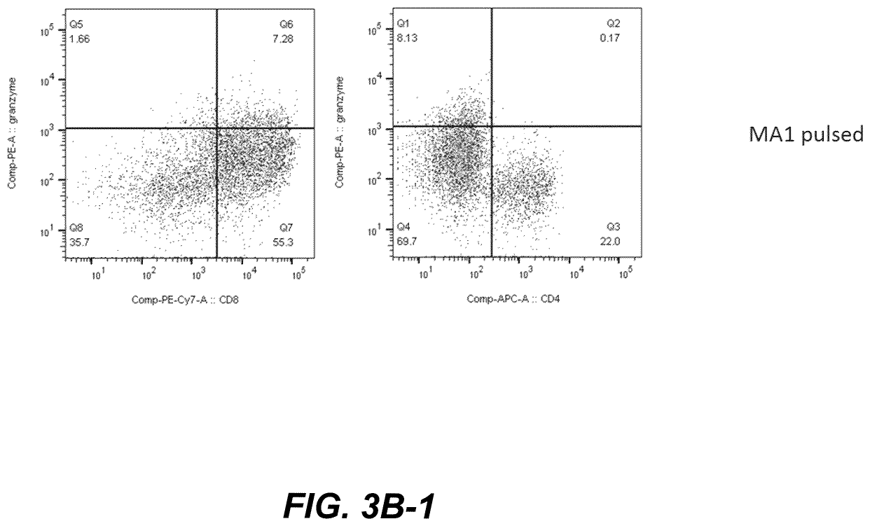

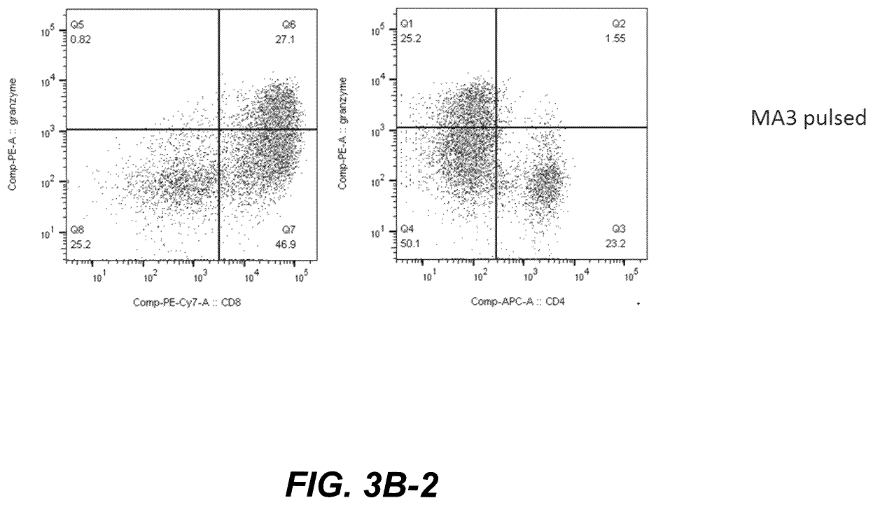

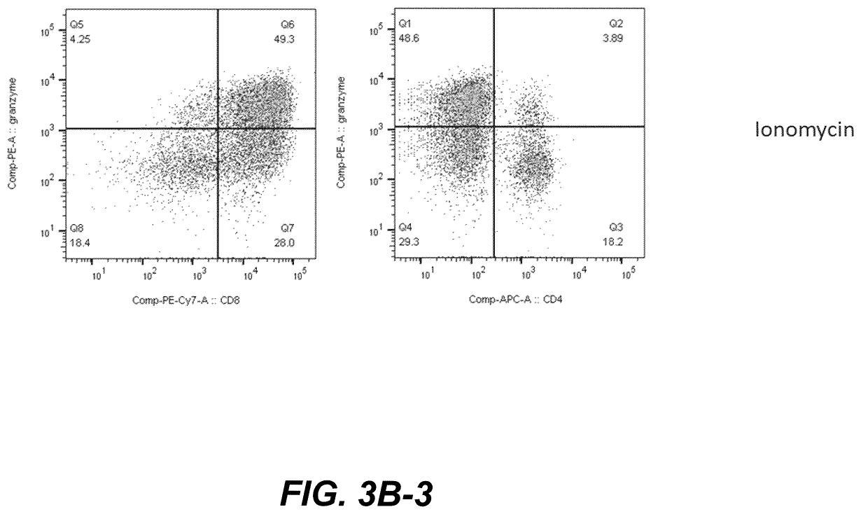

CD4 and CD8 T cells can cause target cell apoptosis through the perforin-granzyme pathway. These components are included in cytoplasmic granules of the effector cells. Upon CD3/TCR activation of T cells the granules are secreted and granzymes and perforin act synergistically to induce apoptosis. To determine whether or not the T cells expressing the MAGE-A-specific CAR of the disclosure lead to T cell activation and apoptosis, a flow cytometric assay was performed. scFv 4A6 CAR T cells were co-incubated for five hours with T2 cells pulsed either with the relevant MA3 peptide or with the irrelevant MA1 peptide. Both peptides show high affinity to HLA-A2 based on Net-MHC prediction. The calcium ionophore, ionomycin, a general T-cell activator was used as a positive control. T cells transduced with pMx-puro RTV014 (not expressing scFv CAR) were used as a negative control. As expected, the positive control, ionomycin, led to high granzyme B production, independently of the type of transduced T cells (bottom panel of FIGS. 3A-1, 3A-2, 3A-3, 3B-1, 3B-2, and 3B-3). Specific activation of scFv 4A6 CAR T cells by T2-MA3 pulsed cells was recorded (FIGS. 3B-1, 3B-2, and 3B-3, middle panel, left dot plot). The activated T cells belong mainly to the CD8-positive fraction, even though there is some minor reactivity by the CD4 subtype.

### EXAMPLE 5

### Purification and Specificity of Bispecific Molecules of the Disclosure Targeting HLA-A2/MAGE-A-Derived Peptides Complexes and CD3

### 5.1 Binding of the Bispecific Molecule of the Disclosure to HLA-A2/MAGE-A-Derived Peptide Complexes

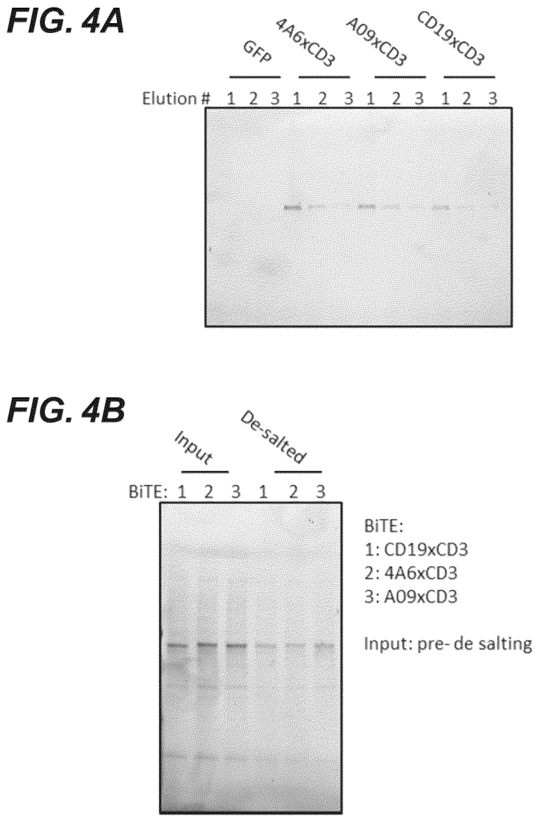

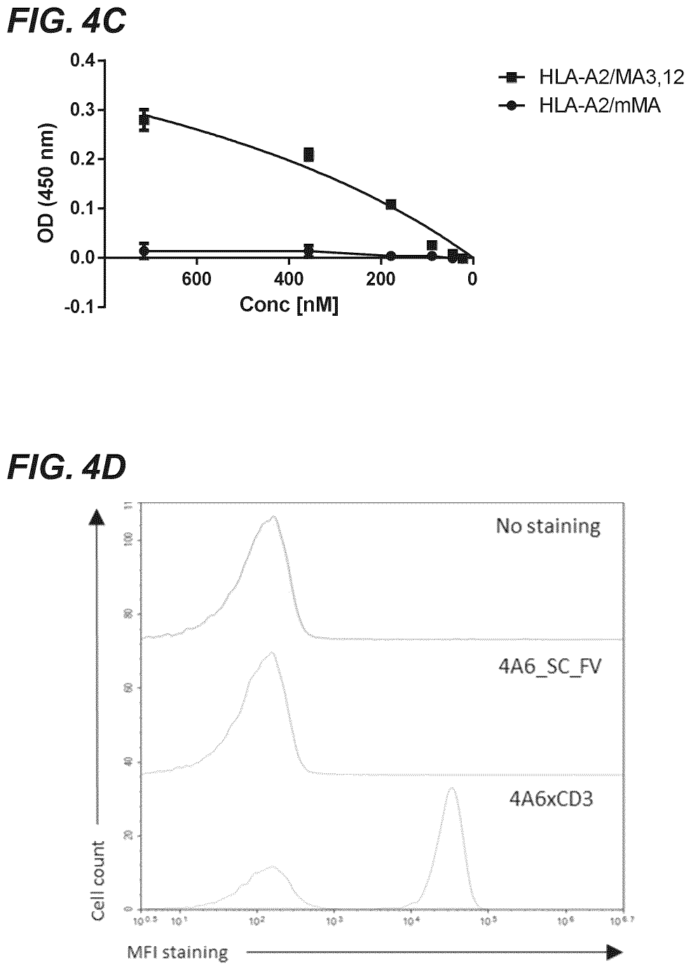

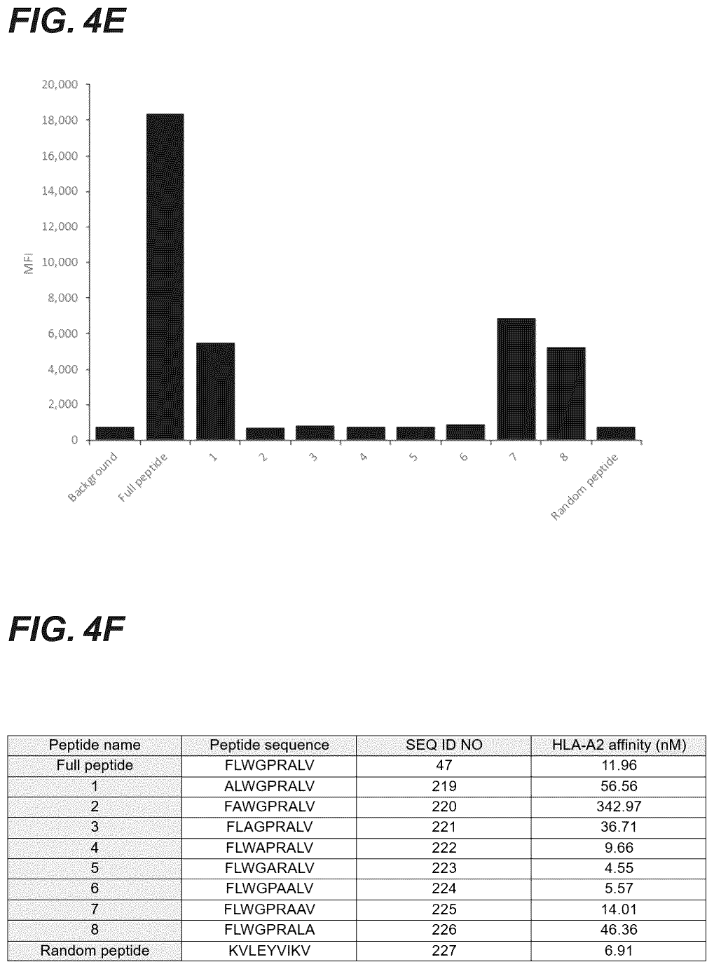

Bispecific molecules were produced in 293F cells transfected with the appropriate pFuse expression vectors at a cell density between 1 and 2 million cells per ml. Transfected cells were allowed to recover for 2 days at 37° C., followed by an incubation at 30° C. for four days during which the bispecific molecules were secreted in the medium. Bispecific molecules were purified from the medium using either Ni-NTA (Thermo Scientific) or Talon beads (Clontech) according to manufacturer's instructions. Upon purification of the molecules, clear bands corresponding to bispecific molecules were visualized on SDS-PAGE as shown in FIGS. 4A and 4B. ELISA assay was used to confirm the specificity of expressed molecules toward HLA-A2/MAGE-A-derived peptide complexes. Biotinylated HLA-A2/MA_{3, 12} (FLWGPRALV (SEQ ID NO:49)) and HLA-A2/mMA (YLEYRQVPG (SEQ ID NO:47)) complexes were coated in a 96-well plate. 4A6xCD3 at decreasing concentration was incubated and allowed to bind the complexes, followed by an incubation with anti-his-HRP antibody. The binding of bispecific molecule was visualized by incubation with 3,3′,5,5′-Tetramethylbenzidine (Thermo Scientific) followed by absorbance measurement at OD450. The results shown in FIG. 4C confirm the specificity of 4A6xCD3 toward HLA-A2/MA_{3, 12}.

### 5.2 Binding of the Bispecific Molecule of the Disclosure to Immune Cells

Binding of 4A6xCD3 to CD3 molecule expressed by T cells was established in a flow cytometric assay by incubating 200.000 peripheral blood mononuclear cells (PBMCs) with 50 ng/ml 4A6xCD3 or 4A6_SC_FV (monospecific antibody fragment used here as a negative control). Flow cytometric analysis showed only binding of 4A6xCD3 to the PBMCs and not of control molecule 4A6_SC_FV (FIG. 4D). 4A6_SC_FV molecule binds specifically to HLA-A2/MA_{3,12} and does not bind CD3 molecule present on immune cells (as shown by lack of signal shift in the middle histogram of FIG. 4D). This result confirmed that 4A6xCD3 specifically recognizes CD3 molecule expressed on the surface of T cells.

### 5.3 Determination of 4A6xCD3 Fine Specificity

Fine specificity of the bispecific molecule was assessed by pulsing 200.000 JY cells overnight under serum free conditions with 100 μg/ml peptide variants. The amino acids of the used peptides were sequentially substituted for an alanine. Pulsed JY cells were incubated with constant concentration of 4A6xCD3. The binding of the 4A6xCD3 was detected upon incubation with anti-his antibody. The obtained binding pattern presented in FIG. 4E showed that all peptide amino acids contribute to the binding of 4A6xCD3 to the HLA-A2/MA_{3,12} complex and that amino acids at positions 2 till 6 were of particular importance for the binding of 4 A6xCD3toward the HLA-A2/MA_{3,12} complex. Table presented in FIG. 4F lists amino acid sequences of all peptides used in this alanine scan experiment, as well as their predicted affinity to HLA-A2.

### EXAMPLE 6

### T-Cell Activation by the Bispecific Molecule of the Disclosure

6.1 Bispecific Molecules of Disclosure Lead to T-Cell Activation in Presence of H1299 Cells Stably Expressing MAGE-A-Derived Peptides in Complex with HLA

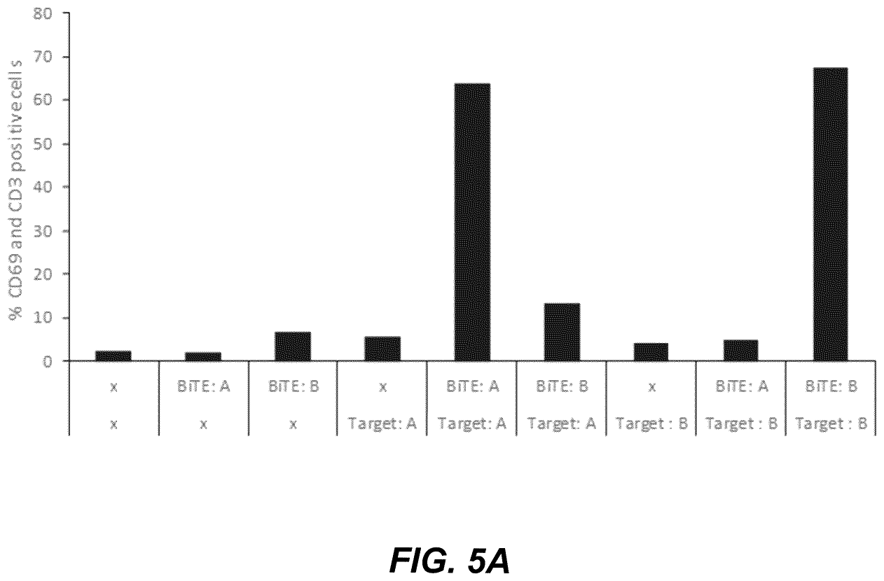

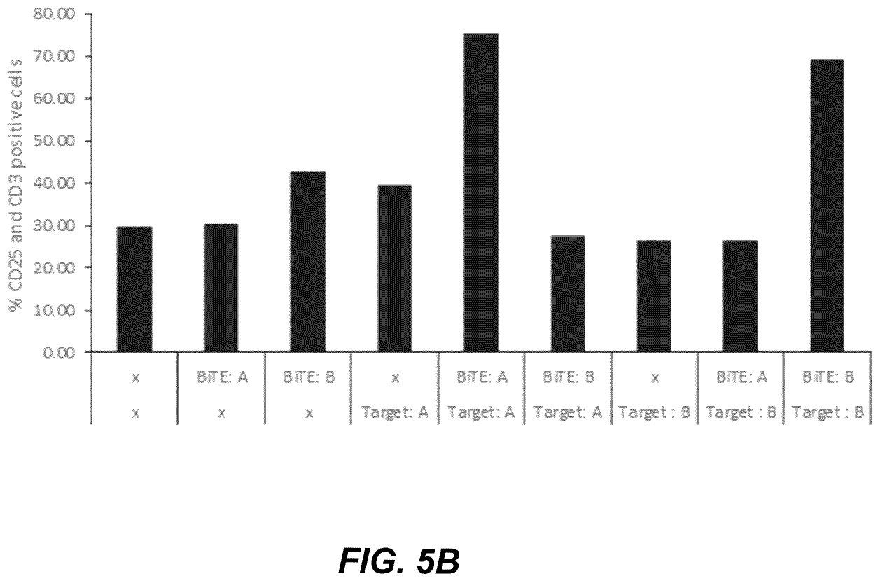

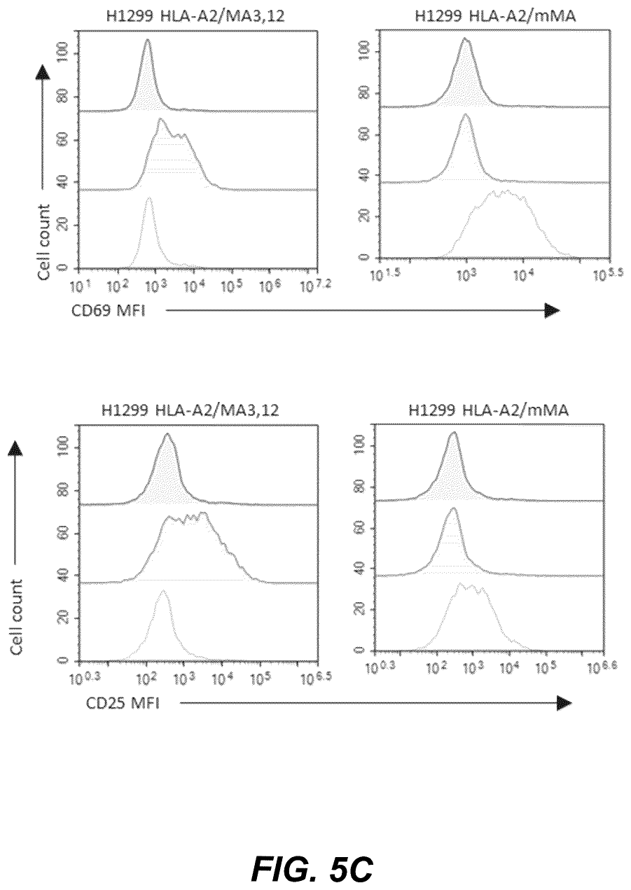

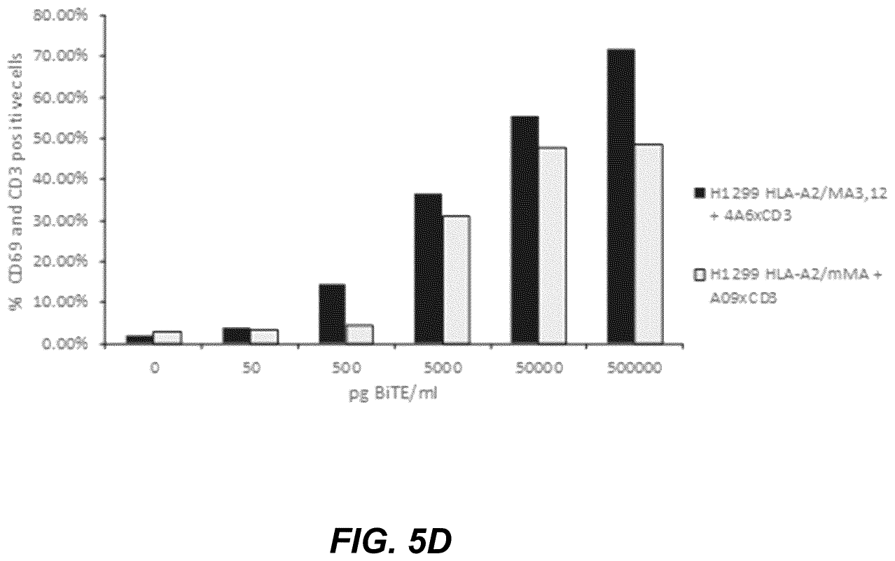

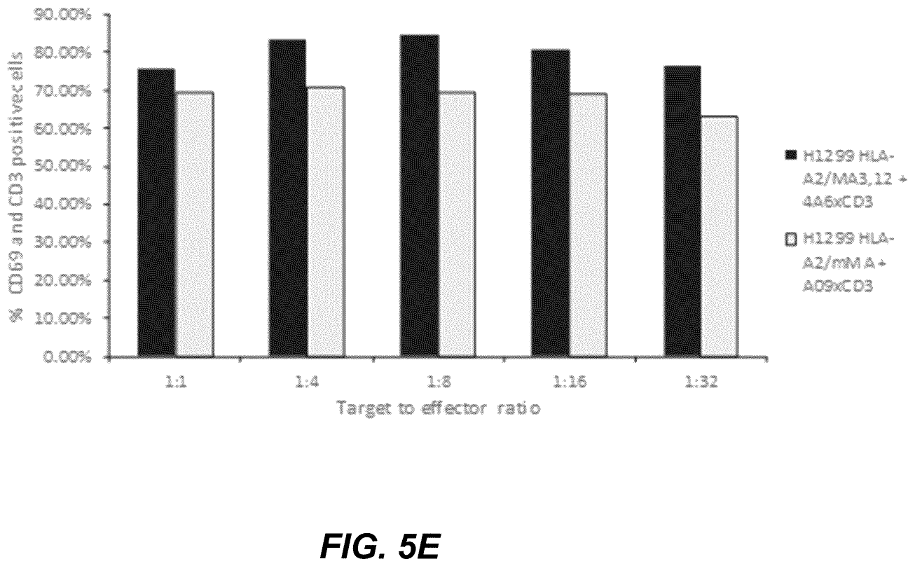

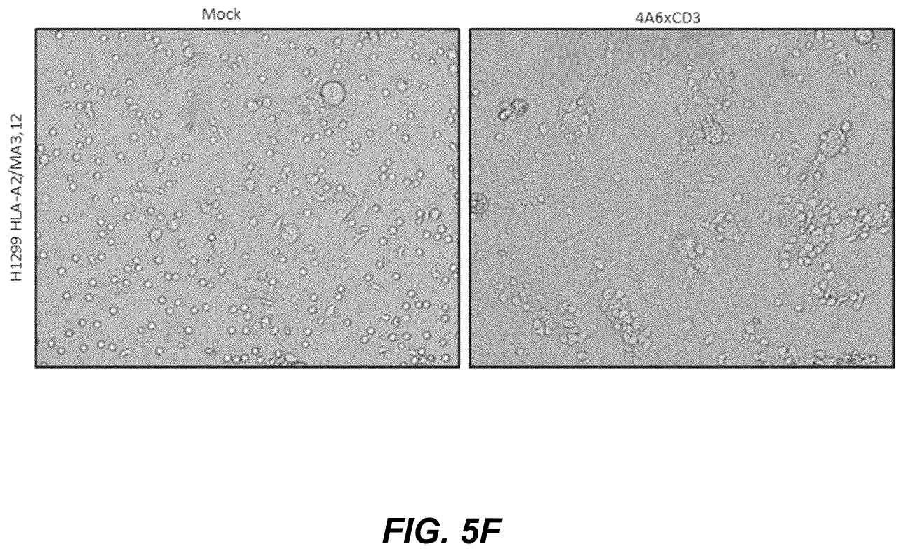

Non-small cell lung carcinoma H1299 cells transfected to stably express respective MAGE-A-derived peptides in complex with HLA, further referred to as target cells, were seeded and allowed to attach to the culture plate overnight. Next day the cell culture medium was refreshed and PBMCs (effector cells) and bispecific molecules of disclosure at concentration of 500 ng/ml were added. The assay was performed at target to effector cells ratio of 1:16 with a 72-hour long incubation. Both target and effector cells were harvested. A flow cytometric analysis was performed in order to detect expression of T-cell activation markers (CD69 and CD25). Results plotted as % of CD3-positive cells expressing CD69 or CD25 are shown in FIGS. 5A and 5B, respectively. Specific increase in both T-cell activation markers was observed only when PBMCs were incubated with bispecific molecules and respective target cells. Incubation of PBMCs and target cells in absence of bispecific molecules did not lead to increase of CD69 or CD25 expression. Histograms presented in FIG. 5C confirm that specific T-cell activation takes place only in presence of bispecific molecules, target cells and PBMCs.

### 6.2 T-Cell Activation is Dependent on Bispecific Molecule Concentration

Respective target cells were seeded and allowed to attach to the culture plate overnight. Next day the cell culture medium was refreshed and PBMCs (effector cells) as well as bispecific molecules of disclosure at increasing concentration were added. The assay was performed at target to effector cells ratio of 1:16 with a 72-hour long incubation. Both target and effector cells were harvested. A flow cytometric analysis was performed in order to detect expression of T-cell activation markers (CD69 and CD25). Specific increase in both T-cell activation markers was observed when PBMCs were incubated with either 4A6xCD3 or A09xCD3 with respective target-expressing cell line (FIG. 5D). Increase of T-cell activation was observed with increase of bispecific molecule's concentration.

### 6.3 Effect of Target to Effector Cells Ratio on T-Cell Activation

When target cells were incubated with a constant concentration of bispecific molecule (500 ng/ml) and varying target to effector ratios for 72 hours (FIG. 5E), no difference in level of T-cell activation determined as expression level of CD69 and CD25 was observed.

### 6.4 Formation of Immune Synapse

Formation of immune synapse was observed upon microscopic inspection of cells used in assays described under 6.1-6.4. The physical attraction of immune cells to target cells shown in FIG. 5F was observed after 24 hours only in the samples that showed increase of T-cell activation markers measured by flow cytometry.

### EXAMPLE 7. BISPECIFIC MOLECULES OF DISCLOSURE LEAD TO T-CELL ACTIVATION

7.1. Bispecific Molecules of Disclosure Lead to T-Cell Activation in Presence of 911 Cells Stably Expressing MAGE-A-Derived Peptides in Complex with HLA

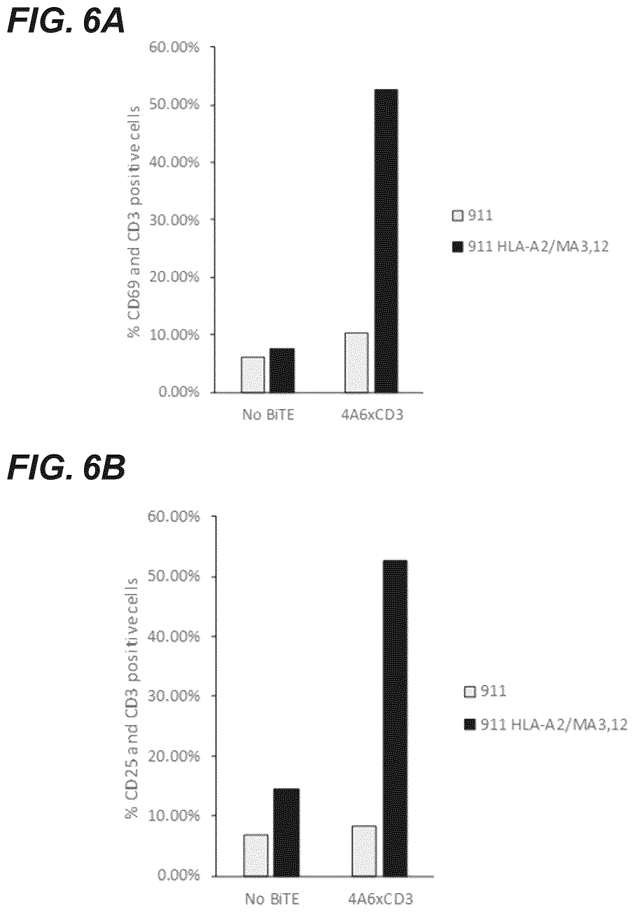

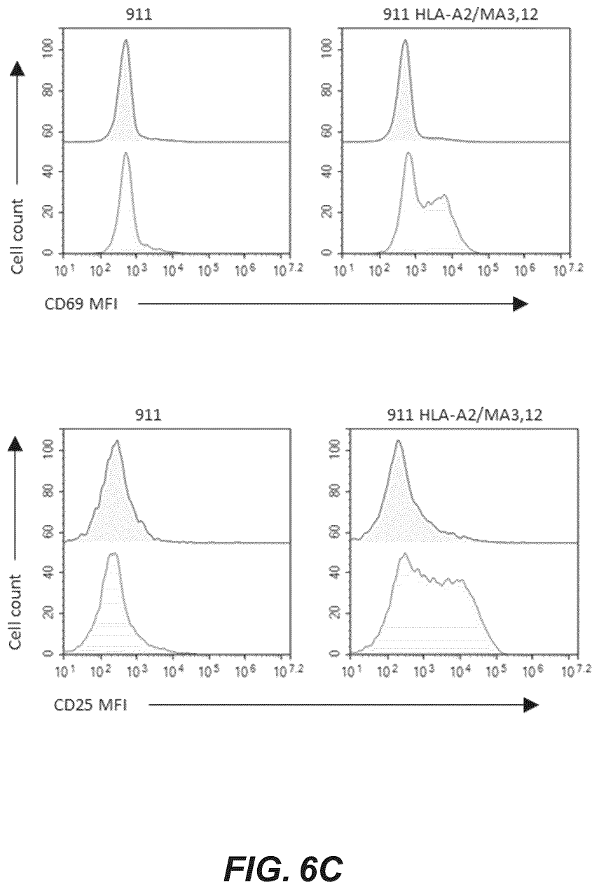

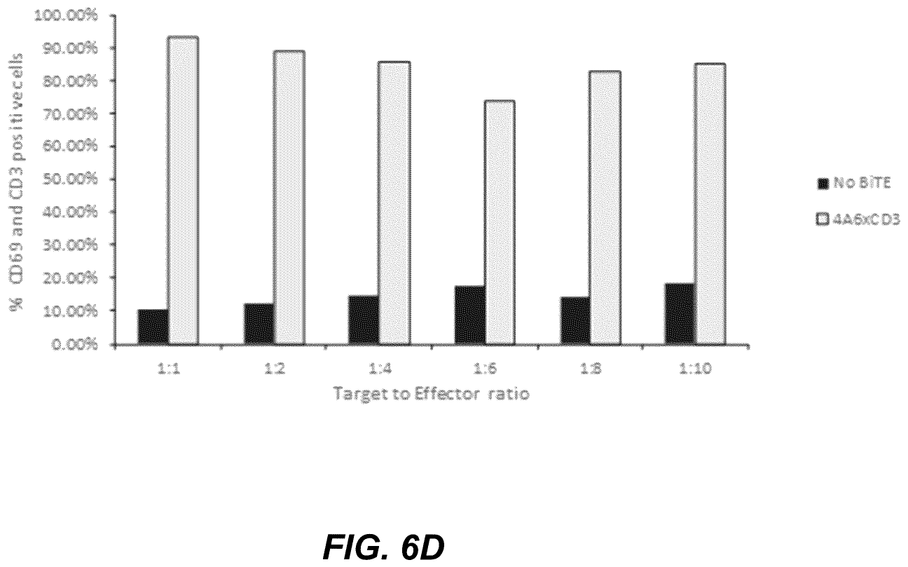

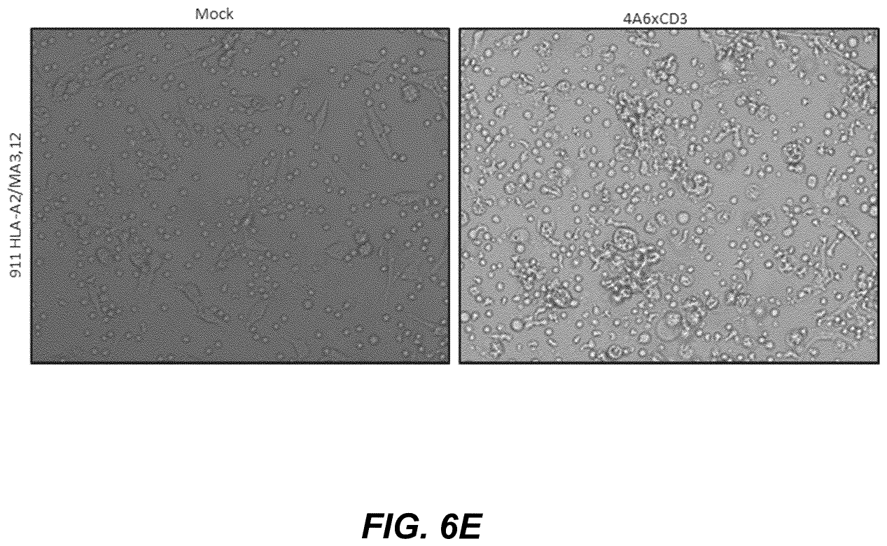

Transformed human embryonic retina cells transfected to stably express respective MAGE-A-derived peptides in complex with HLA, further referred to as target cells, were seeded and allowed to attach to the culture plate overnight. Next day the cell culture medium was refreshed. PBMCs (at a target to effector ratio of 1:8) and 4A6xCD3 (at 500 ng/ml) were added and incubated for 72 hours. Both target and effector cells were harvested. Flow cytometric analysis of effector cells showed increase in expression of T-cell activation markers CD69 and CD25. Results plotted as % of CD3-positive cells expressing CD69 or CD25 are shown in FIGS. 6A and 6B, respectively. Histograms presented in FIG. 6C confirm that specific T-cell activation takes place only in presence of bispecific molecules, target cells and PBMCs, as shown by the clear shift in the MFI signal recorded under those conditions.

### 7.2 Effect of Target to Effector Cells Ratio on T-Cell Activation

When target cells were incubated with a constant concentration of bispecific molecule (500 ng/ml) and varying target to effector ratios for 72 hours, no difference in level of T-cell activation determined as expression level of CD69 and CD25 was observed (FIG. 6D).

During the assays described above target cells could hardly be observed after 72 hours in the conditions showing T-cell activation.

### 7.3 Formation of Immune Synapse

Formation of immune synapse was observed in assays described under 7.1 and 7.2. The physical attraction of immune cells to target cells as shown in FIG. 6E was observed after 24 hours only in the samples that showed increase of T-cell activation markers measured by flow cytometry. Upon a 72-hour incubation of target cells with effector cells in presence of bispecific molecule, hardly any target cells remained.

### EXAMPLE 8

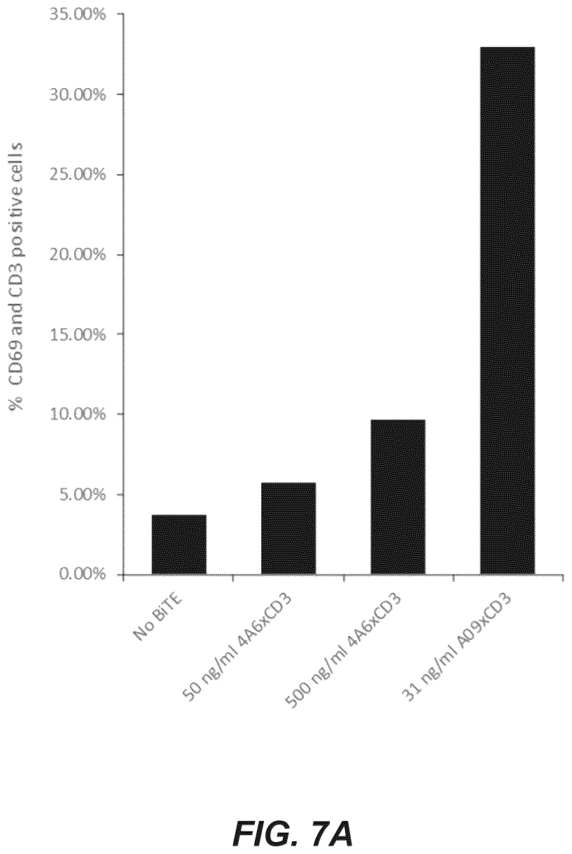

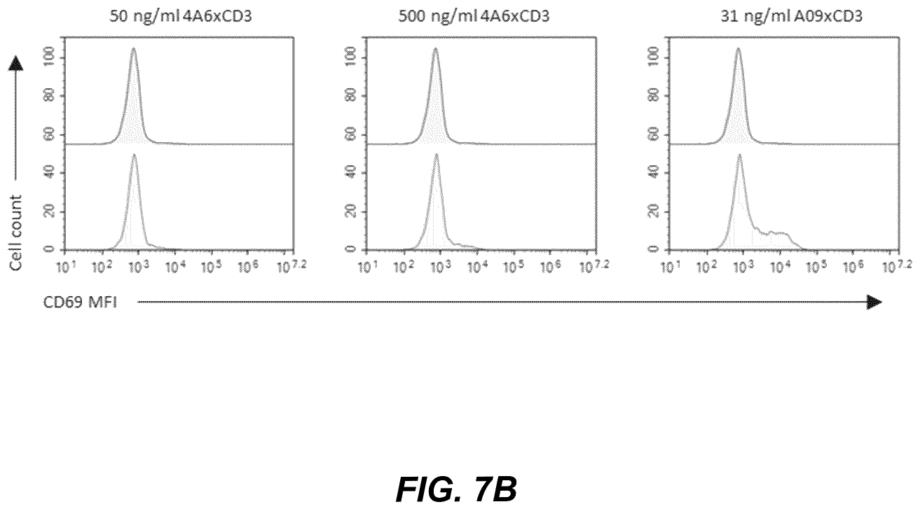

T-cell activation upon incubation with A09xCD3 and glioblastoma cells. U87 cells, which express both MAGE-A and HLA-A2 proteins, were seeded and allowed to attach to culture plate overnight. Next day the culture medium was refreshed. PBMCs were added at target to effector ratio of 1:8, whereas bispecific molecule 4A6xCD3 was added at a final concentration of either 50 ng/ml or 500 ng/ml and A09xCD3 at 31 ng/ml. The incubation lasted for 72 hours. Both target and effector cells were harvested and analyzed by flow cytometry. Expression of T-cell activation marker CD69 was evaluated. Specific increase in expression of T-cell activation markers plotted in FIG. 7A was observed when PBMCs were incubated with A09xCD3 in presence of U87 cells. A clear shift in the MFI signal recorded under those conditions is presented in histograms of FIG. 7B.

### EXAMPLE 9

### Production of Bi-Specific Nanobodies

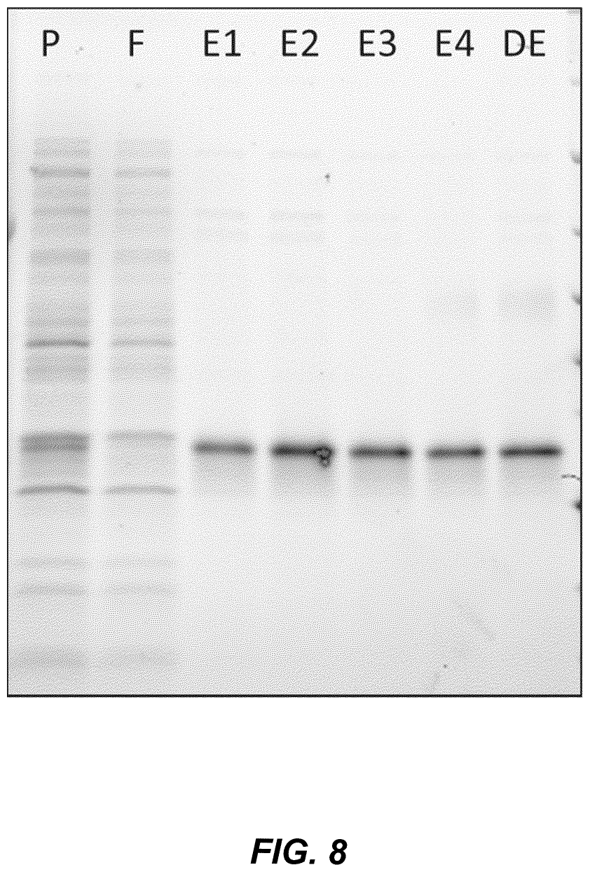

BL21 cells were grown in 2YT medium at 37° C. until a logarithmic growth phase was reached. Isopropyl β-D-1 thioglactopyranoside (IPTG) was added to the medium to a final concentration of 1 mM to induce production of bispecific nanobody molecule. Upon addition of IPTG temperature was decreased to 25° C. and incubation continued for 16 hours. At the end of incubation cells were pelleted by centrifugation (15 minutes at 400 g) and resuspended in PBS. To isolate produced nanobodies bacterial cell pellet was subjected to three freeze thaw cycles. Cellular debris was removed by centrifugation (15 minutes at 4000 g). Supernatant containing produced nanobody was subjected to incubation with NiNTA beads (Thermo Scientific) according to manufacturer's protocol. Efficiency and purity of produced nanobodies was assessed by stain free SDS-PAGE (Biorad) as shown in FIG. 8.

### EXAMPLE 10

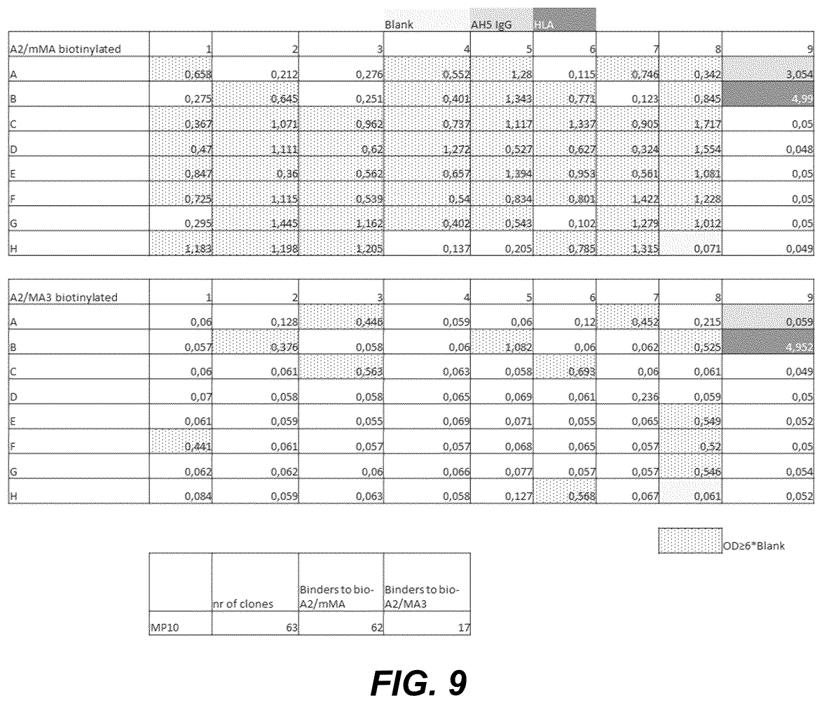

Specific binding of phage display selected Fab fragments to HLA-A2/mMA complexes. Upon affinity driven phage display selection-specific binders were eluted and obtained clones were expressed in bacteria. The periplasmic fractions were isolated and diluted 1:5. Neutravidin (at 2 μg/ml) plates were coated with 10 nM HLA-A2/mMA peptide. The binding of expressed Fab was detected upon incubation with detection antibodies: mouse anti-c-myc (1:1000) and anti-mouse IgG-HRP (1:5000). As a positive control, AH5 Fab (produced from pCES vector) and AH5 monoclonal IgG were used. Binding of produced Fab clones was assessed in parallel on plates coated with HLA-A2/mMA peptide complex and plates coated with control HLA-A2/MA3 peptide complex. Only Fab clones that showed binding to HLA-A2/mMA peptide complex (upper table in FIG. 9) and not to HLA-A2/MA3 peptide complex (bottom table in FIG. 9) were considered to have the desired specificity toward HLA-A2 presenting the multi MAGE peptide (YLEYRQVPG (SEQ ID NO:47)). Clones that showed binding to both types of complexes were considered to be recognizing the HLA-A2 part of the complexes and lack the desired fine specificity. Table 1 provides overview of VH sequences specifically binding MAGE-derived peptides in complex with HLA-A0201. SEQ ID NO:3 through SEQ ID NO:46 represent VH sequence of Fab specifically binding HLA-A2/mMA complex.

TABLE 1

| SEQ ID NO | Description |
|---|---|
| 1 | Vh 4A6 |
| 2 | Vh A09 |
| 3 | MP08A03 |
| 4 | MP08A08 |
| 5 | MP08A09 |
| 6 | MP08B02 |
| 7 | MP08B06 |
| 8 | MP08C01 |
| 9 | MP08C03 |
| 10 | MP08C10 |
| 11 | MP08D02 |
| 12 | MP08D03 |
| 13 | MP08D04 |
| 14 | MP08D07 |
| 15 | MP08D10 |
| 16 | MP08E05 |
| 17 | MP08E06 |
| 18 | MP08E10 |
| 19 | MP08E11 |
| 20 | MP08F02 |
| 21 | MP08F03 |
| 22 | MP08F04 |
| 23 | MP08F05 |
| 24 | MP08F06 |
| 25 | MP08F08 |
| 26 | MP08F09 |
| 27 | MP08G02 |
| 28 | MP08G04 |
| 29 | MP08H01 |
| 30 | MP08H02 |
| 31 | MP08H05 |
| 32 | MP08H09 |
| 33 | MP08H10 |
| 34 | MP09A10 |
| 35 | MP09B10 |
| 36 | MP09C01 |
| 37 | MP09C02 |
| 38 | MP09C03 |
| 39 | MP09C04 |
| 40 | MP09D03 |
| 41 | MP09D09 |
| 42 | MP09E01 |
| 43 | MP09G02 |
| 44 | MP09G03 |
| 45 | MP09G05 |
| 46 | MP09H01 |

What is claimed is:

1. A bispecific antibody comprising a first arm and a second arm, wherein
the first arm specifically binds to an MHC-peptide complex comprising a peptide derived from MAGE associated with aberrant cells, and
the second arm specifically recognizes a target associated with immune effector cells,
wherein the peptide derived from MAGE associated with aberrant cells is derived from YLEYRQVPG (SEQ ID NO:47) or FLWGPRALV (SEQ ID NO:49), and
wherein the bispecific antibody comprises an immunoglobulin variable region comprising a Vh domain comprising SEQ ID NO:1 or SEQ ID NO:2.

2. The bispecific antibody of claim 1, wherein the Vh is in a bi-specific T-cell engager format.

3. The bispecific antibody of claim 1, wherein the bispecific antibody is selected from the group consisting of a human IgG, a human IgG1, and a human IgG wherein the Fc part does not activate the Fc receptor.

4. The bispecific antibody of claim 1, wherein the immune effector cells comprise T cells and NK cells.

5. The bispecific antibody of claim 1, wherein the target associated with an immune effector cell is selected from the group consisting of CD3, CD16, CD25, CD28, CD64, CD89, NKG2D, and NKp46.

6. The bispecific antibody of claim 1, together with pharmaceutically acceptable suitable diluents and/or excipients.

7. The bispecific antibody of claim 1, wherein the target associated with an immune effector cell is CD3.

8. A method of treating a subject diagnosed with cancer, the method comprising:
administering to the subject the bispecific antibody of claim 1 so as to treat the cancer.
